SEQUENCE LISTING (7 sequences)

SEQ ID NO: 1 - protein, 9 aa, homo sapiens
QMRPVSRVL

SEQ ID NO: 2 - protein, 9 aa, Mouse Sp.
QMRPVSRAL

SEQ ID NO: 3 - protein, 9 aa, homo sapiens
VMAPRTVLL

SEQ ID NO: 4 - protein, 14 aa, Clostridium tetani
QYIKANSKFIGITE

SEQ ID NO: 5 - protein, 13 aa, homo sapiens
NFFRMVISNPAAT

SEQ ID NO: 6 - protein, 18 aa, homo sapiens
NPVVHFFKNIVTPRTPPP

SEQ ID NO: 7 - DNA, 31 bp, Artificial Sequence
  primer directed to human HLA-E
ggggatccaacaagctgtgagactcagaccc

(19) United States
(12) Patent Application Publication
(10) Pub. No.: US 2012/0183518 A1
(43) Pub. Date: Jul. 19, 2012

(54) ASSAY FOR DETERMINING HEALTH OF CD8+ T CELLS

(76) Inventors: Hong Jiang, Fort Lee, NJ (US); Leonard Chess, Scarsdale, NY (US)

(21) Appl. No.: 13/392,481

(22) PCT Filed: Aug. 24, 2010
(86) PCT No.: PCT/US10/02341

§ 371 Date: Apr. 2, 2012

Related U.S. Application Data
(60) Provisional application No. 61/340,492, filed on Mar. 18, 2010.
(60) Provisional application No. 61/276,733, filed on Sep. 15, 2009.

(30) Foreign Application Priority Data
Aug. 24, 2009 (US) 12/583723

(51) Int. Cl. A61K 35/14; C12Q 1/25; G01N 21/64; C12Q 1/02

(52) U.S. Cl. 424/93.71; 435/29

(57) ABSTRACT

A method of determining if a subject's CD8+ T-cells functionally recognize an HLA-E/Hsp60sp target structure comprising a) contacting a sample of the subject's CD8+ T-cells with a HLA-E+ cell loaded with Hsp60sp, b) quantifying proliferation of the contacted HLA-E+ cell, c) contacting a sample of the subject's CD8+ T-cells with a HLA-E+ cell which is loaded with a peptide which does not bind to HLA-E, d) quantifying proliferation of the HLA-E+ cell loaded with the peptide which does not bind to HLA-E following contact with the subject's CD8+ T-cells, e) comparing the proliferation quantified in step d) with the proliferation quantified in step b)

This application claims priority of U.S. Provisional Applications Nos. 61/340,492, filed Mar. 18, 2010 and 61/276,733, filed Sep. 15, 2009 and U.S. Ser. No. 12/583,723, filed Aug. 24, 2009, the entire content of each of which is hereby incorporated by reference herein.

Throughout this application, various publications are referenced in parentheses by number. Citations for these references may be found at the end of the specification immediately preceding the claims. The disclosures of these publications in their entireties are hereby incorporated by reference into this application to more fully describe the state of the art to which this invention pertains.

This invention was made with government support under grant nos. R01 AI065609 and U19 AI46132 awarded by the National Institutes of Health. The Government has certain rights in this invention.

### BACKGROUND

How to specifically and effectively prevent and treat human auto-immune diseases remains a central problem in medicine. Part of the reason is because we do not fully understand how the immune system discriminates self from non-self after thymic negative selection in order to maintain peripheral self-tolerance. It is generally accepted that thymic negative selection, in which thymocytes expressing TCR of high avidity for MHC/self-peptide complexes are deleted (1-3) eliminates the periphery and is the major mechanism of self-tolerance. However, release of a large fraction of intermediate avidity self-reactive T cells into the periphery by thymic negative selection represents a potential danger of pathogenic auto-immunity inherited in each individual (4-6), because potentially pathogenic self-reactive T cells are included in the pool of intermediate avidity T cells and can often be functionally activated to elicit auto-immune diseases (6-9). Thus self/non-self discrimination must continue in the periphery after thymic negative selection, and a major function of peripheral regulatory mechanisms is to selectively down-regulate immune responses to self-antigens without damaging the ongoing responses to foreign pathogens (10-12).

In this regard, it has been demonstrated in murine studies that self nonself discrimination is accomplished by thymic negative selection followed by peripheral T cell regulation in which Qa-1 restricted CD8+ T cells selectively down-regulate intermediate avidity T cells activated by both self and foreign antigens (11-15). Unlike Foxp3+CD25+Tregs and any other currently known regulatory cells, which control the magnitude and class of immune responses (16, 17), Qa-1 restricted CD8+ T cells represent the only currently identified regulatory mechanism that functions to discriminate self from non-self in the periphery (11, 12). Since the peripheral self-reactive T cell repertoire does not possess high avidity portion due to thymic negative selection, selective down-regulation of intermediate avidity T cells simultaneously enables the suppression of auto-immunity and the preservation of the functional anti-infection immunity, which is dominated by high avidity T cells (11-15). It has also been demonstrated that a heat shock peptide (Hsp60sp), coupled with the MHC class Ib molecule Qa-1, is a common surrogate target structure, preferentially expressed at a higher level on the intermediate avidity T cells and specifically recognized by the Qa-1 restricted CD8+ T cells (14). This discovery reveals the molecular interaction between the Qa-1 restricted CD8+ T cells and their target T cells and explains why Qa-1 restricted CD8+ T cells are able to selectively down-regulate intermediate avidity T cells. This finding provided a molecular and cellular mechanism that perceiving the avidity of T cell activation can be translated into peripheral T cell regulation to discriminate self from non-self in the periphery (15). Herein a novel assay useful in light of these discoveries is described for determining the health of a subject's HLA-E+ restricted CD8+ T cells.

### SUMMARY OF THE INVENTION

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a B7sp peptide;
d) quantifying proliferation of the HLA-E+ cell which is loaded with the B7sp peptide and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

A method of determining if a subject not known to have an autoimmune disease is predisposed to develop the autoimmune disease comprising determining if the subject's CD8+ T-cells are able to functionally recognize an HLA-E/Hsp60sp target structure on the surface of a cell and inhibit proliferation of the cell, wherein if the subject's CD8+ T-cells are unable to functionally recognize an HLA-E/Hsp60sp target structure on the surface of the cell and inhibit proliferation of the cell then the subject is predisposed to develop the autoimmune disease.

A method of determining if a subject not known to have an autoimmune disease is predisposed to develop the autoimmune disease comprising determining if the subject's HLA-E restricted CD8+ T-cells are able to discriminate self from non-self, wherein if the subject's HLA-E restricted CD8+ T-cells are unable to discriminate self from non-self then the subject is predisposed to develop the autoimmune disease.
A method of determining if a subject's CD8+ T-cells are able to discriminate self from non-self comprising:
   A)
   i) contacting a population of purified CD4+ cells with (a) an amount of a self-antigen and (b) and a population of antigen-presenting cells so as to thereby activate CD4+ cells of the population;
   ii) washing the population of activated CD4+ cells so as to remove the self-antigen;
   iii) culturing a portion of the population of activated CD4+ cells and a portion of the population of antigen-presenting cells together in the presence of a population of the subject's CD8+ T-cells;
   iv) quantifying proliferation of the activated CD4+ cells; and
   v) repeating steps A)i) through A)iv) with different amounts of self-antigen so as to determine the amount of self-antigen required to elicit maximum proliferation of the activated CD4+ cells and thereby determine a self-antigen ED₅₀ for the population of purified CD4+ cells;
   B)
   i) contacting a sample of peripheral blood mononucleocyte cells obtained from the subject with (a) the self-antigen and (b) and a population of antigen-presenting cells so as to thereby activate CD4+ cells of the population;
   ii) washing the population of activated CD4+ cells so as to remove the self -antigen;
   iii) culturing a portion of the population of activated CD4+ cells and a portion of the population of antigen-presenting cells together in the presence of a population of the subject's CD8+ T-cells;
   iv) quantifying proliferation of the activated CD4+ cells;
   v) repeating steps B)i) through B)iv) with different amounts of self-antigen so as to determine the amount of self-antigen required to elicit maximum proliferation of the activated CD4+ cells and thereby determine the self-antigen ED₅₀ for the sample of peripheral blood mononucleocyte cells;
   wherein A) and B) can be performed in any order; and
   C) comparing the self-antigen ED₅₀ and the foreign-antigen ED₅₀,
   wherein a foreign-antigen ED₅₀ greater than the self-antigen ED₅₀ indicates that the subject's CD8+ T-cells are unable to discriminate self from non-self and wherein a foreign-antigen ED₅₀ equal to or lesser than the self-antigen ED₅₀ indicates that the subject's CD8+ T-cells are able to discriminate self from non-self.

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step a); and
e) comparing the proliferation quantified in step d) with the proliferation quantified in step b), wherein,
wherein a greater amount of proliferation quantified in step d) than quantified in step b) indicates that the subject's CD8+ T-cells are able to functionally recognize the HLA-E/Hsp60sp target structure and wherein a lesser amount of proliferation quantified in step d) than quantified in step b) indicates that the subject's CD8+ T-cells are not able to functionally recognize the HLA-E/Hsp60sp target structure.

A method of determining if a subject's HLA-E restricted CD8+ T cells are activated by HLA-E/Hsp60sp comprising:
a) contacting a sample comprising HLA-E restricted CD8+ T cells obtained from the subject with a composition comprising HLA-E/Hsp60sp; and
b) detecting if step (a) results in secretion of an intracellular cytolytic enzyme by an HLA-E restricted CD8+ T cell of the sample,
wherein secretion of an intracellular cytolytic enzyme by an HLA-E restricted CD8+ T cell of the sample indicates that the subject's HLA-E restricted CD8+ T cells are activated by HLA-E/Hsp60sp, and wherein no detectable secretion of an intracellular cytolytic enzyme in step b) indicates that the subject's HLA-E restricted CD8+ T cells are not activated by HLA-E/Hsp60sp.

A method to identify a functioning HLA-E restricted CD8+ T cell in a sample comprising:
a) contacting the sample with a composition comprising HLA-E/Hsp60sp; and
b) detecting if step (a) results in secretion of an intracellular cytolytic enzyme in a cell of the sample,
wherein secretion of an intracellular cytolytic enzyme in a cell of the sample indicates that the sample comprises a functioning HLA-E restricted CD8+ T cell.

A method for prophylactically treating a subject against developing an autoimmune disease comprising administering to the subject dendritic cells loaded with Hsp60sp peptide so as to activate HLA-E restricted CD8+ T cells in the subject and thereby prophylactically treat the subject against developing the autoimmune disease.

A method for treating a subject having an autoimmune disease comprising administering to the subject dendritic cells loaded with Hsp60sp peptide so as to activate HLA-E restricted CD8+ T cells in the subject and thereby treat the autoimmune disease.

A method of treating a subject having an autoimmune disease comprising:
a) determining if HLA-E restricted CD8+ T cells obtained from the subject are able to discriminate self from the non-self; and
b) if the subject's HLA-E restricted CD8+ T cells are determined in step a) as not able to discriminate self from the non-self, administering to the subject an exosome or dentritic cell loaded Hsp60sp peptide so as to activate HLA-E restricted CD8+ T cells in the subject and thereby treat the autoimmune disease.

### BRIEF DESCRIPTION OF THE FIGURES

FIG. 1A. Surface expression of HLA-E on a HLA-E transfectant B721/E after loading with peptides. HLA-E transfectants were loaded with peptide followed the surface staining with anti-HLA-E mAb as described in the methods.

FIG. 1B. HLA-E restricted CD8+ T cells [CD8(H)] specifically inhibit HLA-E expressing cells loaded with Hsp60sp. CD8(H) and control lines were generated and tested in a CD8+ T cell inhibition assay as described in the methods. Data was from three healthy normal individuals.

FIG. 1C. HLA-E restricted CD8+ T cells suppress the overall immune responses to self-antigen MBP and GAD But enhance the immune responses to foreign antigen TT and PPD. CD8(H) and control lines were generated and tested in a self/nonself discrimination assay as described in the methods. Data was from three healthy normal individuals.

FIG. 2A. CD8+ T cells in the freshly isolated PBMC from a Type 1 Diabetes (T1D) patient lost the capacity to discriminate self from nonself, compared with normal individual. CD8+ T cells were purified from PBMC from T1D patients and tested in a CD8+ T cell inhibition assay as described in the methods. Data shown is representative of 9 out of 10 T1D patients tested paired with healthy normal controls.

FIG. 2B. CD8+ T cells in the freshly isolated PBMC from a T1D patient lost the capacity to specifically recognize HLA-E/Hsp60sp target structure, compared with normal individual. CD4+ T cells were purified from PBMC from T1D patients and tested and compared with PBMC in a standard self/nonself discrimination assay as described in the methods. Data shown is representative of 9 out of 10 T1D patients tested paired with healthy normal controls.

FIG. 3A. CD8+ T cells restored the capacity to discriminate self from nonself after boosted in vitro with autologous dendritic (DCs) loaded with Hsp60sp peptide, compared with normal individual. CD8(H) and control CD8+ lines were generated from each T1D patient and corresponding normal controls and tested and compared in a self/nonself discrimination assay as described. Data shown is representative of 8 out of 9 T1D patients that originally tested with defect of the HLA-E restricted CD8+ T cells, paired with healthy normal controls.

FIG. 3B. CD8+ T cells restored the capacity to specifically recognize the target structure HLA-E/Hsp60sp, after boosted in vitro with autologous DCs loaded with Hsp60sp peptide, compared with normal individual. CD8(H) and control CD8+ lines were generated from each T1D patient and corresponding normal controls and tested and compared in a CD8+ T cell inhibition assay as described. Data shown is representative of 8 out of 9 T1D patients that originally tested with defect of the HLA-E restricted CD8+ T cells, paired with healthy normal controls.

FIG. 3C. CD8+ T cells in freshly isolated PBMC from majority of the T1D patients tested lost the capacity to discriminate self from nonself in the periphery compared with normal control people. Immune responses of purified CD4+ T cells to self-antigen GAD versus to foreign antigen TT were compared with PBMC in each T1D patient, paired with normal control. SND Index is used as a parameter to evaluate the differences between the responses to self versus to foreign antigens in each individual in the presence and absence of CD8+ T cells. Data is representative of two tests for each patient.

FIG. 3D. CD8+T cells from most diabetic patients tested regain the capacity to discriminate self from nonself after an in vitro boost. Immune responses of in vitro established CD8(H) lines to self-antigen GAD versus to foreign antigen TT were compared with CD8(B) and CD8(N) lines in each T1D patient, paired with normal control. SND Index is used as a parameter to evaluate the differences between the responses to self versus to foreign antigens in each individual. Data is representative of two tests for each patient.

FIG. 4. Freshly isolated CD8+ T cells from majority of the T1D patients tested lost the capacity to discriminate self from nonself in the periphery. Immune responses of purified CD4+ T cells to self-antigen MBP versus to foreign antigen TT were compared with CD4+ T cells plus CD8+ T cells in each T1D patient, paired with normal control. Data summarizes 10 T1D patients and corresponding controls.

FIG. 5. Increased cytolytic enzyme (CE) expression by the HLA-E restricted CD8+ T cells triggered by the specific target structure HLA-E/Hsp60sp. CD8(H) and CD(B) lines from healthy individuals were co-cultured with B721/E cells loaded with peptide Hsp60sp or control peptide B7sp. The CE expression by the CD8+ T cells was detected by a three color intracellular staining with Perforin (Perf), Granzyme A (GA) and Granzyme B (GB) followed by FACS analysis as described in the method. Data were representative of data from one of three healthy normal individuals. Upper panel represents the CE Expression Indexes of three different combinations of CE expression and lower panel shows the intracellular staining patterns of the CEs on the CD8(H) lines.

### DETAILED DESCRIPTION OF THE INVENTION

As used herein “HLA-E” has the common meaning as used in the art, i.e. human leukocyte antigen system E.

HLA-E protein sequences are described by NCBI accession nos. CAA05527, CAA40172, BAB63328, and BAF31260, hereby incorporated by reference. In embodiments, the agent is a HLA-E/IgG fusion protein, the agent is a HLA-E tetramer or HLA-E/Hsp60sp tetramer. Tetramers are described in, for example, Salcedo et al., Eur. J. Immunol. 2000 Apr;30(4):1094-101, which is hereby incorporated by reference. The avidity model and intermediate avidity T-cells are described in WO/2008/103471 published Aug. 28, 2008, which is hereby incorporated by reference.

As used herein a “HLA-E restricted CD8+ T cell” is a regulatory CD8+ T cell that recognizes the peptides presented by HLA-E molecule on the immune system antigen presenting cells (APC) or on HLA-E+ dendritic cells. An antigen presenting cell for the HLA-E restricted CD8+ T cells as encompassed herein includes an intermediate avidity T cell, which is also a specific target for these CD8+ T cells.

As used herein a cell or membrane bound composition “loaded” with a peptide shall mean that the cell or membrane bound composition has been incubated with the peptide under conditions permitting entry into and/or attachment onto the cell or membrane bound composition of the peptide. For example, dendritic cells can be loaded with Hsp60sp or B7sp by incubating the cells with Hsp60sp or B7sp, respectively, at a concentration of 50 uM, and a temperature of 37° C., for 2 hours.

In embodiments, the HLA-E⁺ cell is a CD4⁺/HLA-E⁺ T cell, a CD8⁺/HLA-E⁺ T cell.

In a preferred embodiment the Hsp60sp is human. Human Hsp60sp is QMRPVSRVL (SEQ ID NO:1). Murine Hsp60sp has the sequence QMRPVSRVL (SEQ ID NO:2). B7sp has the sequence VMAPRTVLL (SEQ ID NO:3).

A “self-antigen” is defined with regard to the organism in which it is being described and is a physiological constituent of the organism's own tissues and body components capable of stimulating autoimmunity.

A “foreign-antigen” or a nonself antigen defined with regard to the organism in which it is being described and is an entity which is not a physiological constituent of the organism's own tissues and body components and which is capable of stimulating an immune response in the organism.

Proliferation of cells as used herein can be quantified in a manner known in the art.

“ED₅₀” as used herein shall mean the dose of antigen or self-antigen that elicits half-maximal proliferation of the pertinent cells, for example antigen-activated CD4+ T cells.

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a B7sp peptide;
d) quantifying proliferation of the HLA-E+ cell which is loaded with the B7sp peptide and contacted with the subject's CD8+ T-cells in step c);
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

In an embodiment of the instant methods the sample of the subject's CD8+ T-cells in step a) and/or in step c) is a peripheral blood mononucleocyte cell sample obtained from the subject. In an embodiment of the instant methods the subject has an autoimmune disease. In an embodiment of the instant methods the autoimmune disease is type 1 diabetes.

A method of determining if a subject not known to have an autoimmune disease is predisposed to develop the autoimmune disease comprising determining if the subject's CD8+ T-cells are able to functionally recognize an HLA-E/Hsp60sp target structure on the surface of a cell and inhibit proliferation of the cell, wherein if the subject's CD8+ T-cells are unable to functionally recognize an HLA-E/Hsp60sp target structure on the surface of the cell and inhibit proliferation of the cell then the subject is predisposed to develop the autoimmune disease.

In an embodiment of the instant method, whether the subject's CD8+ T-cells are able to functionally recognize an HLA-E/Hsp60sp target structure on the surface of the cell and inhibit proliferation of the cell is determined by:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser amount of proliferation quantified in step d) than quantified in step b) indicates that the subject's CD8+ T-cells are not able to functionally recognize the HLA-E/Hsp60sp target structure.

A method of determining if a subject not known to have an autoimmune disease is predisposed to develop the autoimmune disease comprising determining if the subject's HLA-E restricted CD8+ T-cells are able to discriminate self from non-self, wherein if the subject's HLA-E restricted CD8+ T-cells are unable to discriminate self from non-self then the subject is predisposed to develop the autoimmune disease.

In an embodiment of the instant method, determining if a subject's CD8+ T-cells are able to discriminate self from non-self comprises:
A)
i)contacting a population of CD4+ cells with (a) an amount of a self-antigen and (b) and a population of antigen-presenting cells so as to thereby activate CD4+ cells of the population;
ii) washing the population of activated CD4+ cells so as to remove the self-antigen;
iii) culturing a portion of the population of activated CD4+ cells and a portion of the population of antigen-presenting cells together in the presence of a population of the subject's CD8+ T-cells;
iv) quantifying proliferation of the activated CD4+ cells; and
v) repeating steps A)i) through A)iv) with different amounts of self-antigen so as to determine the amount of self-antigen required to elicit maximum proliferation of the activated CD4+ cells and thereby determine a self-antigen ED₅₀;
B)
i) contacting a population of CD4+ cells with (a) a foreign-antigen and (b) and a population of antigen-presenting cells so as to thereby activate CD4+ cells of the population;
ii) washing the population of activated CD4+ cells so as to remove the foreign-antigen;
iii) culturing a portion of the population of activated CD4+ cells and a portion of the population of antigen-presenting cells together in the presence of a population of the subject's CD8+ T-cells;
iv) quantifying proliferation of the activated CD4+ cells;
v) repeating steps B)i) through B)iv) with different amounts of foreign-antigen so as to determine the amount of foreign-antigen required to elicit maximum proliferation of the activated CD4+ cells and thereby determine a foreign-antigen ED₅₀;
wherein A) and B) can be performed in any order; and
C) comparing the self-antigen ED₅₀ and the foreign-antigen ED₅₀,
wherein a foreign-antigen ED₅₀ greater than the self-antigen ED₅₀ indicates that the subject's CD8+ T-cells are unable to discriminate self from non-self and wherein a foreign-antigen ED₅₀ equal to or lesser than the self-antigen ED₅₀ indicates that the subject's CD8+ T-cells are able to discriminate self from non-self.

In an embodiment of the instant method the CD8+ T-cells are contained in a sample of peripheral blood mononucleocyte cells obtained from the subject. In an embodiment of the instant method the CD8+ T-cells are HLA-E restricted.

A method of determining if a subject's CD8+ T-cells are able to discriminate self from non-self comprising:
A)
i) contacting a population of purified CD4+ cells with (a) an amount of a self-antigen and (b) and a population of antigen-presenting cells so as to thereby activate CD4+ cells of the population;
ii) washing the population of activated CD4+ cells so as to remove the self-antigen;
iii) culturing a portion of the population of activated CD4+ cells and a portion of the population of antigen-presenting cells together in the presence of a population of the subject's CD8+ T-cells;
iv) quantifying proliferation of the activated CD4+ cells; and
v) repeating steps A)i) through A)iv) with different amounts of self-antigen so as to determine the amount of self-antigen required to elicit maximum proliferation of the activated CD4+ cells and thereby determine a self-antigen ED₅₀ for the population of purified CD4+ cells;
B)
i) contacting a sample of peripheral blood mononucleocyte cells obtained from the subject with (a) the self-antigen and (b) and a population of antigen-presenting cells so as to thereby activate CD4+ cells of the population;
ii) washing the population of activated CD4+ cells so as to remove the self-antigen;
iii) culturing a portion of the population of activated CD4+ cells and a portion of the population of antigen-presenting cells together in the presence of a population of the subject's CD8+ T-cells;
iv) quantifying proliferation of the activated CD4+ cells;
v) repeating steps B)i) through B)iv) with different amounts of self-antigen so as to determine the amount of self-antigen required to elicit maximum proliferation of the activated CD4+ cells and thereby determine the self-antigen ED₅₀ for the sample of peripheral blood mononucleocyte cells;
wherein A) and B) can be performed in any order; and
C) comparing the self-antigen ED₅₀ and the foreign-antigen ED₅₀,
wherein a foreign-antigen ED₅₀ greater than the self-antigen ED₅₀ indicates that the subject's CD8+ T-cells are unable to discriminate self from non-self and wherein a foreign-antigen ED₅₀ equal to or lesser than the self-antigen ED₅₀ indicates that the subject's CD8+ T-cells are able to discriminate self from non-self.

A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step a);
e) comparing the proliferation quantified in step d) with the proliferation quantified in step b), wherein,
wherein a greater amount of proliferation quantified in step d) than quantified in step b) indicates that the subject's CD8+ T-cells are able to functionally recognize the HLA-E/Hsp60sp target structure and wherein a lesser amount of proliferation quantified in step d) than quantified in step b) indicates that the subject's CD8+ T-cells are not able to functionally recognize the HLA-E/Hsp60sp target structure.

In an embodiment of the instant methods the sample of the subject's CD8+ T-cells in step a) and/or in step c) is a peripheral blood mononucleocyte cell sample obtained from the subject. In an embodiment of the instant methods the subject has an autoimmune disease. In an embodiment of the instant methods the autoimmune disease is type 1 diabetes.

A method of determining whether a subject is likely to develop an autoimmune disorder comprising determining if a subject's CD8+ T cells are not able to discriminate self from the non-self, wherein a subject whose CD8+ T cells are not able to discriminate self from non-self is determined as likely to develop an autoimmune disease. In an embodiment the subject's CD8+ T cells are determined as able to discriminate self from non-self by a method described hereinabove.

A method of determining if a subject suffering from an autoimmune disease can be treated for the autoimmune disease by administration of HSP60sp-loaded cells comprising determining if the subject's CD8+ T cells functionally recognize an HLA-E/Hsp60sp target structure, wherein a subject suffering from the autoimmune disease whose CD8+ T cells are not able to functionally recognize the HLA-E/Hsp60sp target structure is determined as treatable by administration of Hsp60sp-loaded cells. In an embodiment the subject's CD8+ T cells are determined as able to functionally recognize the HLA-E/Hsp60sp target structure by self by a method described hereinabove.

A method of determining if a subject's HLA-E restricted CD8+ T cells are activated by HLA-E/Hsp60sp comprising:
a) contacting a sample comprising HLA-E restricted CD8+ T cells obtained from the subject with a composition comprising HLA-E/Hsp60sp; and
b) detecting if step (a) results in secretion of an intracellular cytolytic enzyme by an HLA-E restricted CD8+ T cell of the sample,
wherein secretion of an intracellular cytolytic enzyme by an HLA-E restricted CD8+ T cell of the sample indicates that the subject's HLA-E restricted CD8+ T cells are activated by HLA-E/Hsp60sp, and wherein no detectable secretion of an intracellular cytolytic enzyme in step b) indicates that the subject's HLA-E restricted CD8+ T cells are not activated by HLA-E/Hsp60sp.

In an embodiment, the composition comprising HLA-E/Hsp60sp is a cell transfected to express HLA-E and loaded with Hsp60sp. In an embodiment, the cell transfected to express HLA-E is a B721 cell. In an embodiment, the intracellular cytolytic enzyme is perforin, granzyme A or granzyme B. In an embodiment, the sample is a blood sample or is derived from blood. In an embodiment, the cytolytic enzyme is detected by contacting the sample with an anti-cytolytic enzyme antibody conjugated to a detectable marker.

A method to identify a functioning HLA-E restricted CD8+ T cell in a sample comprising:
a) contacting the sample with a composition comprising HLA-E/Hsp60sp; and
b) detecting if step (a) results in secretion of an intracellular cytolytic enzyme in a cell of the sample,
wherein secretion of an intracellular cytolytic enzyme in a cell of the sample indicates that the sample comprises a functioning HLA-E restricted CD8+ T cell.

A method for prophylactically treating a subject against developing an autoimmune disease comprising administering to the subject dendritic cells loaded with Hsp60sp peptide so as to activate HLA-E restricted CD8+ T cells in the subject and thereby prophylactically treat the subject against developing the autoimmune disease.

A method for treating a subject having an autoimmune disease comprising administering to the subject dendritic cells loaded with Hsp60sp peptide so as to activate HLA-E restricted CD8+ T cells in the subject and thereby treat the autoimmune disease.

A method of treating a subject having an autoimmune disease comprising:
a) determining if HLA-E restricted CD8+ T cells obtained from the subject are able to discriminate self from the non-self; and
b) if the subject's HLA-E restricted CD8+ T cells are determined in step a) as not able to discriminate self from the non-self, administering to the subject an exosome or dentritic cell loaded Hsp60sp peptide so as to activate HLA-E restricted CD8+ T cells in the subject and thereby treat the autoimmune disease.

In an embodiment, the autoimmune disease is type 1 diabetes, rheumatoid arthritis, multiple sclerosis, psoriasis, scleroderma, systemic lupus erythematosus. In embodiments the autoimmune disease is alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, crest syndrome, Crohn's disease, Dego's disease, dermatomyositis, dermatomyositis—juvenile, discoid lupus, essential mixed cryoglobulinemia, fibromyalgia —fibromyositis, Grave's disease, Guillain-Barre, Hashimoto's thyroiditis, idiopathic pulmonary fibrosis, idiopathic thrombocytopenia purpura (ITP), IGA nephropathy, insulin dependent diabetes (type I), juvenile arthritis, lupus, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, takayasu arteritis, temporal arteritis/giant cell arteritis, ulcerative colitis, uveitis, vasculitis, vitiligo, or Wegener's granulomatosis.

In an embodiment of the methods, the Hsp60sp peptide has the sequence set forth in SEQ ID NO:1. In an embodiment of the methods, the dendritic cells are immature dendritic cells.

In an embodiment of the instant methods the subject is a human.

All combinations of the various elements of methods, compositions and processes described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

Herein, experimental evidence is provided that HLA-E restricted CD8+ T cells, capable of differentially regulating immune responses to self versus to foreign antigens, can be isolated from healthy individuals that are involved in the development and control of human auto-immune disease Type 1 Diabetes (T1D). Moreover, assays to determine the health of HLA-E restricted CD8+ T cells are also provided and tested.

### Establishment of Human HLA-E Restricted CD8+ T Cell Lines That Specifically Recognize HLA-E/Hsp60sp Expressed on the Target Cells

In previous studies in mice it has been demonstrated that a heat shock peptide (Hsp60sp), coupled with the MHC class Ib molecule Qa-1, is a common surrogate target structure, preferentially expressed on the intermediate avidity T cells activated by any antigens, and specifically recognized by the Qa-1 restricted CD8+ T cells. Thus, by a specific recognition of the common target structure on the intermediate avidity T cells, the Qa-1 restricted CD8+ T cells are able to selectively down-regulate intermediate but not high avidity T cells to accomplish self/nonself discrimination in the periphery (14, 15).

To study the HLA-E restricted human CD8+ T cells, it was confirmed that HLA-E restricted CD8+ T cell lines could be generated from PBMC of healthy humans that specifically recognize Hsp60sp associated with HLA-E. In this regard, in previous studies, it was shown that murine dendritic cells loaded with Qa-1 binding peptide Hsp60sp but not Qdm can be used as a vaccine to induce a CD8+ T cell dependent protection from EAE (14). This observation strongly suggested that functional Qa-1/HLA-E restricted CD8+ T cells could be induced by Hsp60sp loaded dendritic cells in vivo. Employing this approach, two types of human CD8+ T cell lines have been successfully generated in vitro from several DR4+ healthy individuals by stimulating purified peripheral CD8+ T cells with autologous dendritic cells loaded with Hsp60sp, termed “CD8(H)”, or B7sp as control, termed “CD8(B)”.

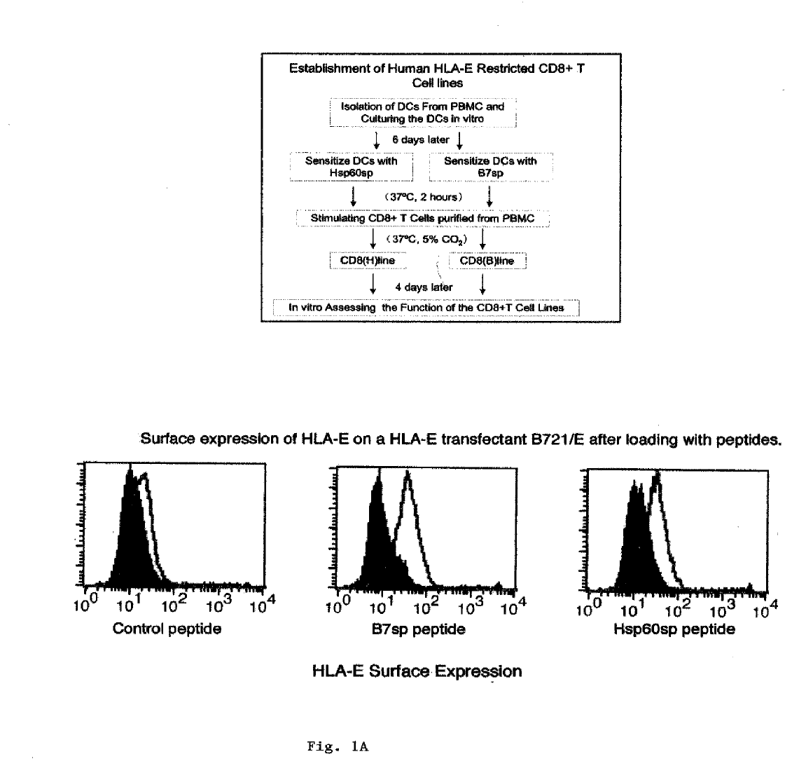

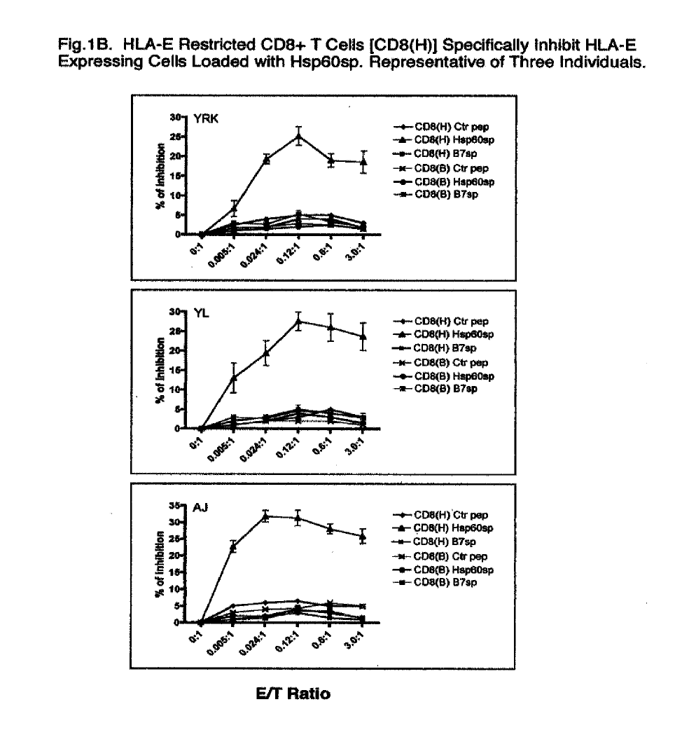

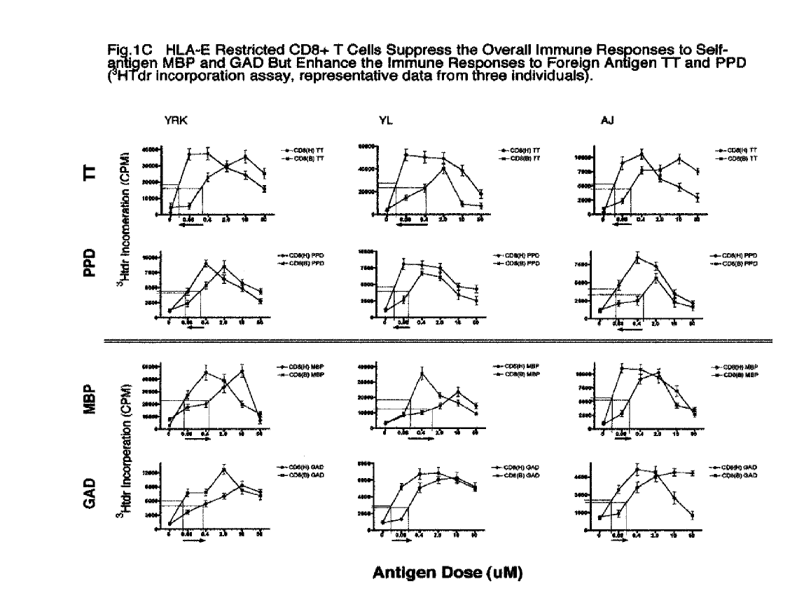

The CD8+ T cell lines generated from the healthy donors were first tested to confirm that they recognize HLA-E/Hsp60sp expressed on the target T cells. For this, human cell lines were established that express HLA-E on their surface in order to identify the HLA-E binding peptide/s that could be recognized by the regulatory CD8+ T cells. Thus, a HLA-E expression construct was established and transfected into a human HLA-A, B, C deficient B cell line B721 (18, 19). HLA-E expression clones were generated by limiting dilution and used as a HLA-E presenting cells to identify the target HLA-E binding peptide/s and test the function of the HLA-E restricted CD8+ T cells. The cloned B721/HLA-E transfectants (B721/E) were first tested for their surface expression of HLA-E as described (14, 20) by staining the peptide loaded B721/E cells with HLA-E specific mAb 3D-12 (21). FIG. 1A shows a representative result of a subclone of B721/E, B721/E, in which both B7sp and Hsp60sp could bind to HLA-E to stabilize the expression of HLA-E on the cell surface.

Hsp60sp Specific CD8+ T Cells Inhibit HLA-E Expressing Cells Loaded with Hsp60sp but not Control Peptide B7sp

The specificity of the CD8(H) lines generated from the healthy individuals was investigated by testing the inhibitory effect of the CD8(H) lines on the HLA-E expressing transfectants B721/HLA-E loaded with Hsp60sp, B7sp or control HLA-E non-binding peptide (CD8+ T cell inhibition assay). Representative results shown in FIG. 1B, CD8+ T cells, triggered by Hsp60sp loaded DC, CD8(H), consistently showed in all the individual tested, a potent suppression on HLA-E transfectants loaded with Hsp60sp but not B7sp or control peptide, compared with the CD8+ T cells triggered by DC loaded with B7sp, CD8(B). The control CD8(B) did not suppress any transfectants. The specific cognitive recognition of HLA-E/Hsp60sp expressed on the target cells by the HLA-E restricted CD8+ T cells was further confirmed by the detection of cytolytic enzymes (CEs) secreted by the HLA-E restricted CD8+ T cells. Thus, increased secretion of CEs, such as perforin, granzyme A and granzyme B by the CD8+ T cells was observed when the CD8(H) lines were co-cultured with B721/E cells loaded with Hsp60sp but not with B721/E cells loaded with control peptide B7sp, or when CD8(B) lines were co-cultured with B721/E loaded with Hsp60sp or B7sp, as shown by representative results in FIG. 5. These observations were precisely correlated with the specific killing of targets by the CD8(H) lines that loaded with Hsp60sp but not B7sp, as detected in the CD8+ T cell inhibition assay above.

These results indicated that, HLA-E restricted, Hsp60sp specific CD8+ T cells, the human counterpart of Qa-1 restricted regulatory CD8+T cells identified in mice, can be generated from the blood of healthy humans.

### HLA-E Restricted CD8+ T Cells Function to Discriminate Self From Non-Self in the Periphery

The effect of the CD8+ T cell lines was tested on the overall avidity of immune responses of CD4+ T cells to self antigens MBP (or GAD) versus to foreign antigens TT (or PPD) in a standard T cell proliferation assay that successfully established and used in previous studies in mice (15). Thus, purified autologous CD4+ T cells were activated by a series dose of either MBP (and GAD) or TT (and PPD), and CD8(H) cells were added into the CD4+ T cell cultures. CD8(B) and “CD8(N)” cells (i.e. primed with DCs without being loaded with peptides) serve as controls. In the studies using GAD as a self-antigen, the donors were selected to be DR4+. As shown by FIG. 1C in the same individuals shown in FIG. 1B, the CD8(H) suppress the immune responses of CD4+ T cells to self-antigens MBP and GAD but enhance the CD4+ T cell immune responses to foreign antigens TT and PPD, compared with CD8(B) or CD8(N)*cells. The typical pattern of a inhibited immune response was shown by a decreased overall avidity, reflected by an increased ED₅₀, in immune responses to self-antigen MBP and GAD in the presence of CD8(H) compared with the responses to the same antigens in the presence of control CD8(B) and CD8(N) cells. Whereas the typical pattern of an enhanced immune response was shown by an increased overall avidity, reflected by a decreased ED₅₀, in immune responses to foreign antigen TT and PPD in the presence of CD8(H) compared with the responses to the same antigens in the presence of control CD8(B) and CD8(N) cells. This set of experiments directly and unequivocally demonstrated that HLA-E restricted, Hsp60sp specific, CD8+ T cells capable of differentially regulating the immune responses to self-antigens versus to foreign antigens in the periphery function in healthy humans.

### A Defect in HLA-E Restricted CD8+ T Cell Mediated Pathway Has Been Detected in Majority of the T1D Patients Tested

In previous studies, it has been demonstrated that Qa-1 restricted CD8+ T cells control the spontaneously developed T1D in NOD mice by means of self/nonself discrimination (15). To confirm that HLA-E restricted CD8+ T cells also play a major role in control of human T1D, the function of the CD8+ T cells in freshly isolated MPBC from 10 T1D patients was tested. The standard antigens chosen are TT for foreign antigen and GAD and MBP for self-antigens, thus in all the experiments throughout this study each patient tested was selected as DR4+ and paired with a DR4+ healthy normal control.

a). CD8+ T Cells Freshly Isolated From Most of the T1D Patients Tested Lost the Capacity to Discriminate Self From Nonself in the Periphery Compared with Normal Control People

First, a T cell proliferation assay was used to evaluate the effect of CD8+ T cells on the CD4+ T cell responses to self-antigen GAD or MBP versus to foreign antigen TT to assess their function of self/nonself discrimination, as described above. In this regard, to determine whether normal human CD8+ T cells can function in vitro to discriminate self from non-self, the overall immune responses of human PBMC was compared for both self and foreign antigens in the presence or absence of CD8+ T cells. In the first set of experiments, an intact PBMC population was cultured containing (a) both the CD8+ T cells and CD4+ T cells, and a population of purified CD4+ T cells was cultured with varying doses of the GAD (and MBP) or TT. In this regard, it was recognized that comparing the PBMC population with the purified CD4+ T cell population alone could not definitively pinpoint the function of the CD8+ T cells contained in the PBMC. However, after large-scale experiments were performed using purified CD8+ T cells as regulators (represented by FIG. 1C) to show that HLA-E restricted CD8+ T cells are capable of discriminating self from non-self, comparing the immune response of PBMC versus purified CD4+ T cells could then be used as a quick read out for the function of the CD8+ T cells in combination with other assays. This is the case in testing the function of CD8+ T cells in T1D patients as described for the initial step and combining such with a CD8+ T cell inhibition assay.

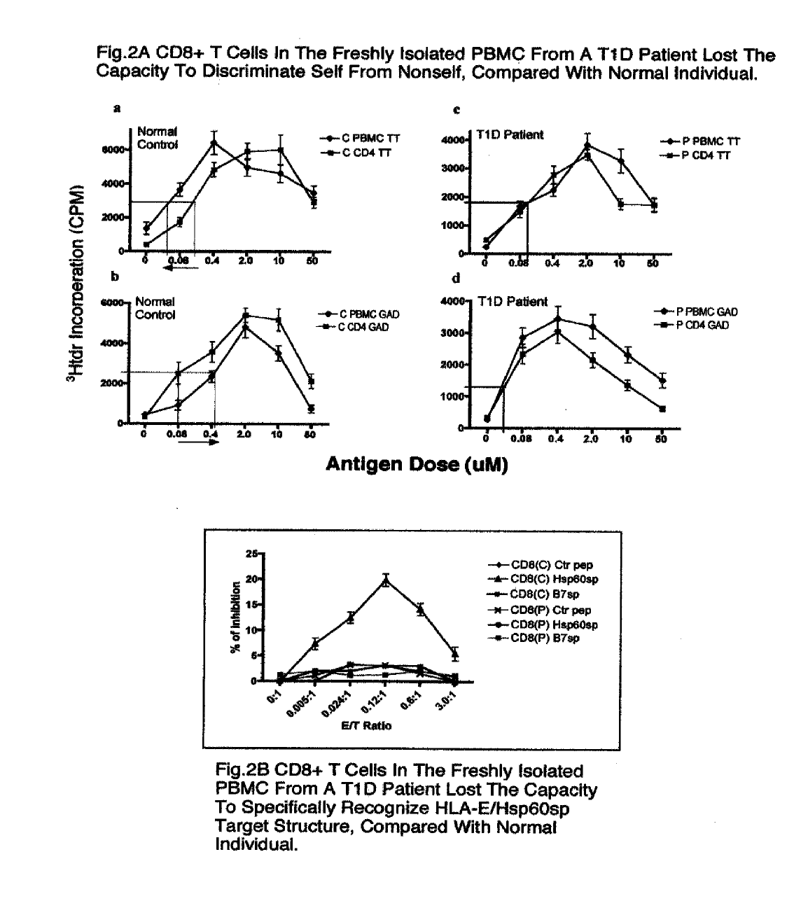

FIG. 2 shows that the presence of CD8+ T cells in PBMC from normal controls significantly depressed the overall response to the self-antigens GAD and MBP, reflected by an increased ED₅₀ (FIG. 2Ab), while enhancing the overall response to a foreign antigen, TT, reflected by a decreased ED₅₀ (FIG. 2Aa), compared with purified CD4+ T cells. However, when the same tests were performed on the PBMC from T1D patients, there was no significant difference of ED₅₀ between PBMC and purified CD4+ T cells responding to either the self-antigens GAD and MBP, or to the foreign antigen TT (FIG. 2Ac and 2Ad), indicating that CD8+ T cells from T1D patients lost the capacity to discriminate self from nonself.

For a better and clearer understanding of the huge amount of functional data obtained by self/nonself discrimination assays in 10 patients and corresponding controls, an additional simpler but meaningful and reliable parameter other than ED₅₀ for each individual to evaluate the function of T cells was needed. In this regard, it is disclosed herein that, in all the normal individuals tested, in the absence of the HLA-E restricted CD8+ T cells the antigen dose required to elicit the highest proliferation of T cells is always higher for the foreign antigen (TT or PPD) than for the self-antigen (GAD or MBP). This pattern is reversed when the HLA-E restricted CD8+ T cells are present in the CD4+ T cell cultures in the normal controls.

Statistical analysis was performed on data from 6 normal controls and 10 T1D patients. Highly significant differences were seen between the antigen doses needed to elicit proliferation in response to the self-antigens GAD and MBP versus the antigen doses needed to elicit maximal proliferation in response to the foreign antigens TT and PPD when in the presence of the regulatory CD8+ T cells in all normal controls. This was observed in both freshly isolated lymphocytes and in in vitro primed CD8 T cells. However, no difference was seen in the self and foreign antigen doses required to elicit maximal proliferation in freshly isolated lymphocytes in most of the T1D patients, regardless of the presence or absence of the CD8+ T cells (Table 2).

To capture this observation a new parameter was conceived—the Self Nonself Discrimination Index (SND Index). The SND is the correlation of antigen doses that elicit the highest amount of T cell proliferation in response to self-antigens versus immune responses to foreign antigens in each individual.

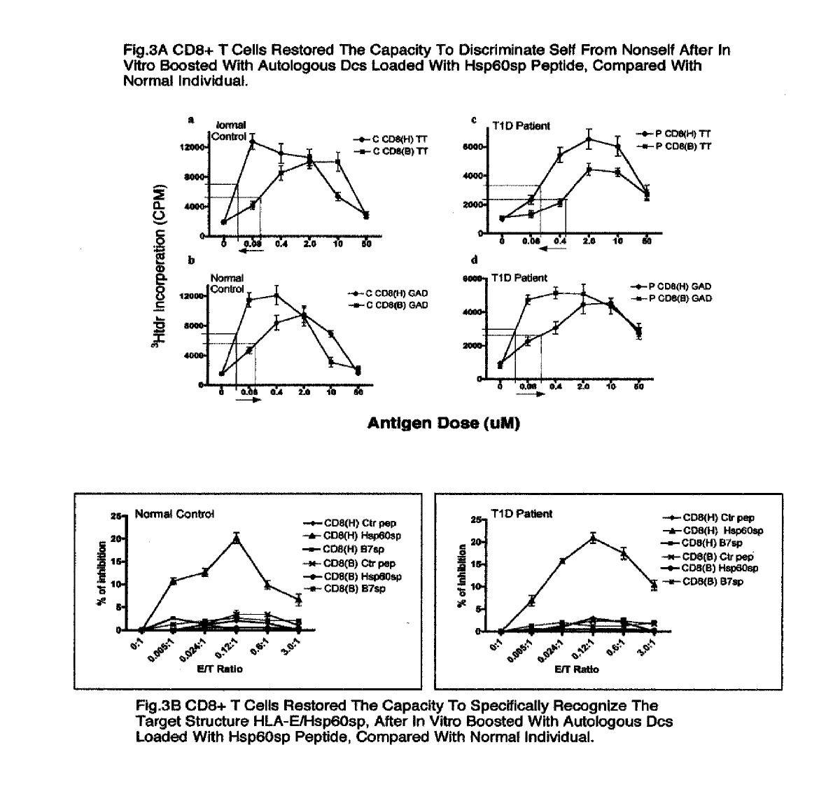

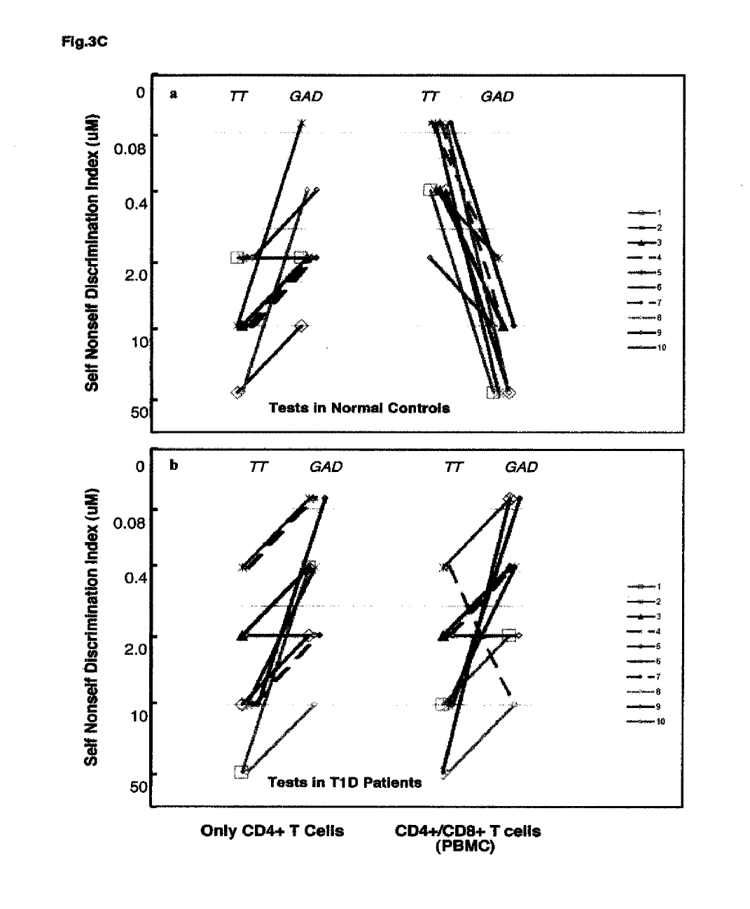

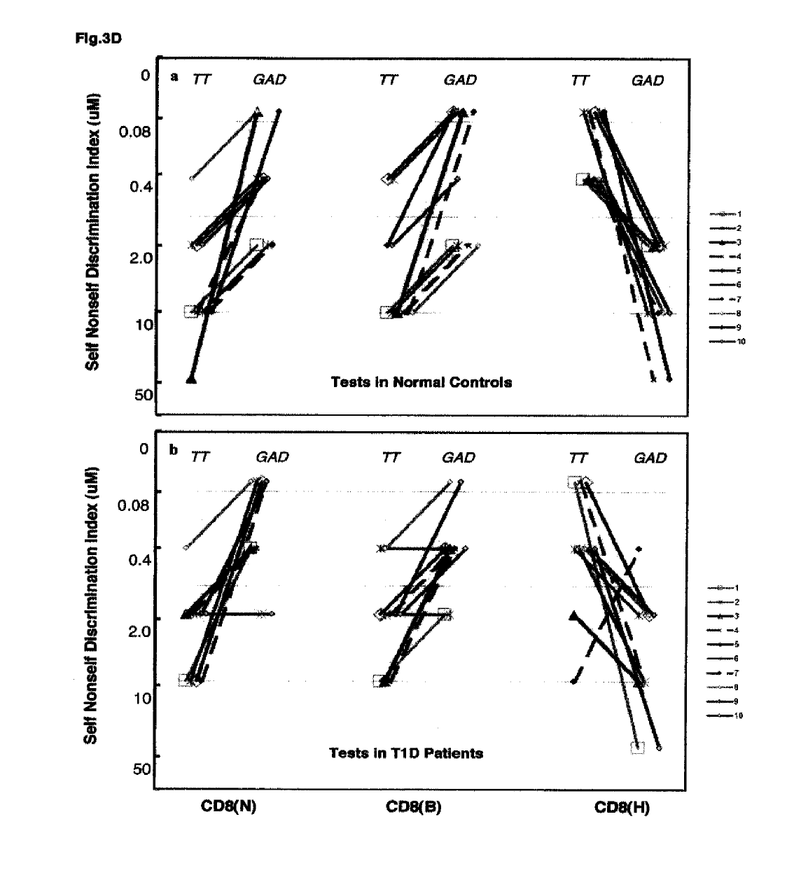

Results from 10 T1D patients and 6 normal controls (in 10 tests) using SND Index as a parameter are summarized in FIG. 3C. Thus, in the CD4+ T cell cultures, lacking the CD8+ T cells, the SND Indexes are not different (P>0.05) for foreign antigen TT responses versus self-antigen GAD responses in both T1D patients and in normal controls (See Table 1). However, consistent with representative results shown in FIG. 2Aa and 2Ab, in the PBMC cultures containing CD8+ T cell all the normal controls showed a different pattern—the SND Indexes of self-antigen GAD responses are increased and significantly higher than those for foreign antigen TT responses. The foreign antigen doses required to elicit maximal proliferation were comparatively dramatically decreased in all individuals tested (P<0.05). This indicates a well-controlled self-reactivity in the normal healthy subjects.

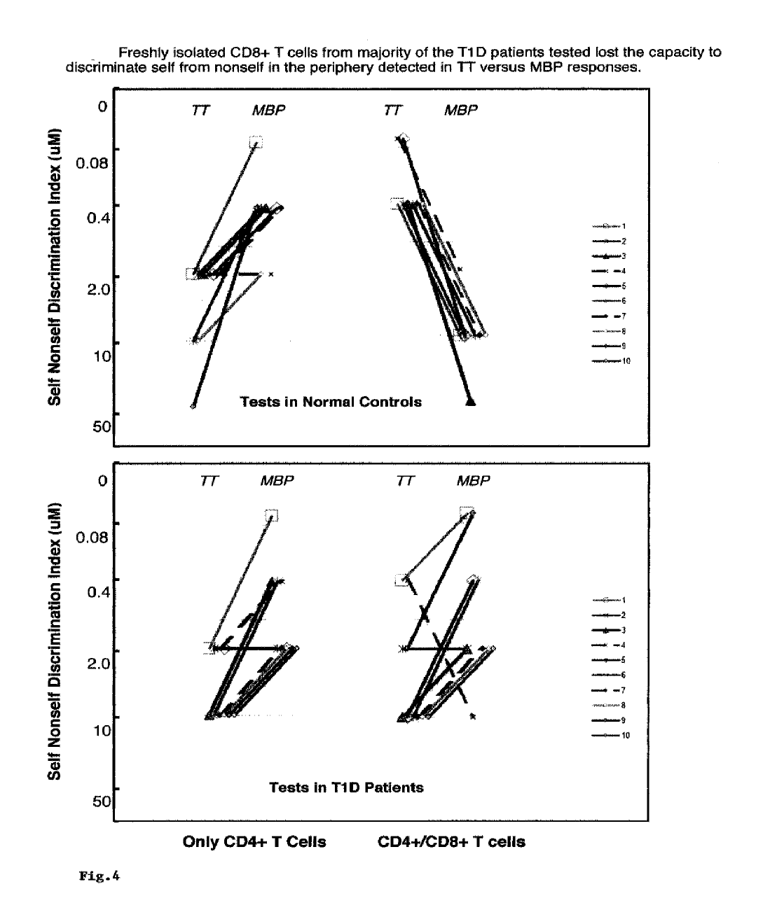

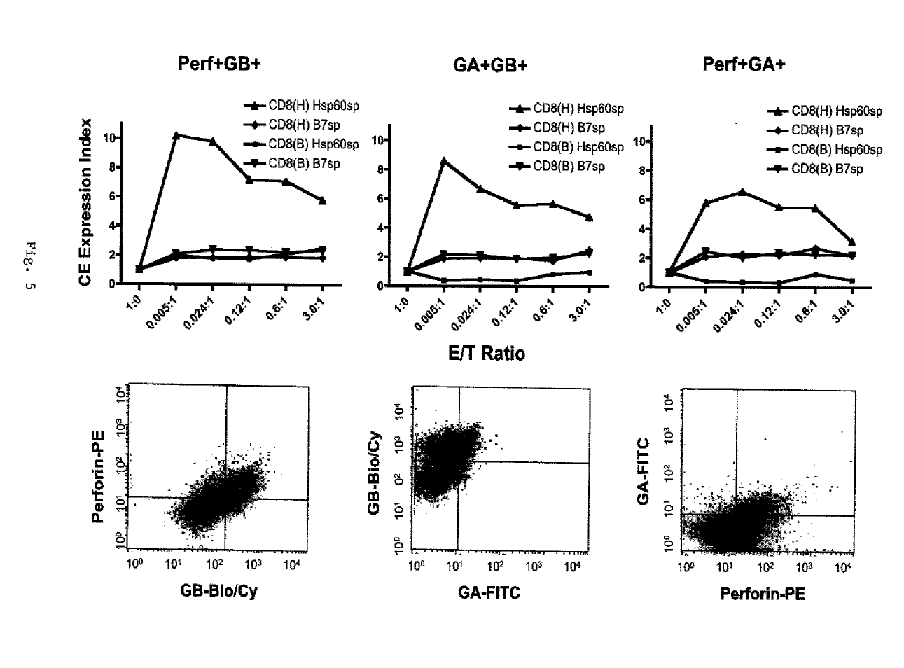

In contrast, 9 out of 10 T1D patients tested showed the same pattern of SND Index's distribution in both the presence of CD8+ T cells and the absence of CD8+ T cells (P>0.05) (except #4 patient). This indicates an uncontrolled self-reactivity to the self antigen GAD in these 9 patients (Table 2). This observation was further substantiated by an increased reactivity to another self-antigen (MBP) in the 9 T1D patients (See FIG. 4 and Table). These observations are consistent with the notion that in the majority of the T1D patients tested there is a defect in the CD8+ T cells that function to discriminate self from nonself.

TABLE 1
T-test of responses to foreign antigen TT versus self-
antigen GAD in freshly isolated CD4+ T cells and
CD8+ cells, i.e. PBMC (T1D patients and normal controls).
TTEST 1

| Within T1D Patients | TT versus GAD |
|---|---|
| CD4+ T | *P = 0.0002 |
| CD4/CD8 T Cells | *P = 0.012 |
| | CD4+ T versus CD4/CD8 T Cells |
| TT | P = 0.87 |
| GAD | P = 0.17 |

TTEST 2

| Within Normal Controls | TT versus GAD |
|---|---|
| CD4+ T | *P = 0.0078 |
| CD4/CD8 T Cells | P = 0.026 |
| | CD4+ T versus CD4/CD8 T Cells |
| TT | P = 0.002 |
| GAD | P = 0.033 |

TTEST 3

| Patients versus Controls | CD4+ T versus CD4+ T |
|---|---|
| TT | P = 0.52 |
| GAD | P = 0.21 |
| | CD4/CD8 T Cells versus |
| | CD4/CD8 T Cells |
| TT | P = 0.002 |
| GAD | P = 0.041 |

The T-test was performed using SND Index as a parameter to analyze the data obtained from self/nonself discrimination assays.
*P is significant in a reversed direction, e.g., SND Indexes are higher for foreign antigen TT than self-antigen GAD.

TABLE 2
T-test of responses to TT versus to GAD in the presence
and absence of regulation by HLA-E restricted CD8+
T cells between T1D patients and normal controls.
TTEST 1

| Within T1D Patients | TT versus GAD |
|---|---|
| CD8(N) | *P = 0.014 |
| CD8(B) | *P = 0.025 |
| CD8(H) | P = 0.041 |
| | CD8(N) versus CD8(H) |
| TT | P = 0.002 |
| GAD | P = 0.039 |
| | CD8(B) versus CD8(H) |
| TT | P = 0.018 |
| GAD | P = 0.043 |

TTEST 2

| Within Normal Controls | TT versus GAD |
|---|---|
| CD8(N) | *P = 0.045 |
| CD8(B) | *P = 0.001 |
| CD8(H) | P = 0.026 |
| | CD8(N) versus CD8(H) |
| TT | P = 0.035 |
| GAD | P = 0.031 |
| | CD8(B) versus CD8(H) |
| TT | P = 0.001 |
| GAD | P = 0.032 |

TTEST 3

| Patients versus Controls | CD8(N) versus CD8(N) |
|---|---|
| TT | P = 0.256 |
| GAD | P = 0.545 |
| | CD8(B) versus CD8(B) |
| TT | P = 0.234 |
| GAD | P = 0.474 |
| | CD8(H) versus CD8(H) |
| TT | P = 0.253 |
| GAD | P = 1.0 |

*P is significant in a reversed direction, i.e. SND Indexes are higher for foreign antigen TT than self-antigen GAD.

TABLE 3
TTEST of responses to TT Versus to MBP from freshly
isolated CD4+ T cells and CD4+ T cells
plus CD8+ T cells between T1D patients and normal controls
TTEST 1

| Within T1D Patients | TT versus MBP |
|---|---|
| CD4+ T | *P = 0.007 |
| CD4/CD8 T Cells | *P = 0.019 |
| | CD4+ T versus CD4/CD8 T Cells |
| TT | P = 0.86 |
| MBP | P = 0.23 |

TTEST 2

| Within Normal Controls | TT versus MBP |
|---|---|
| CD4+ T | *P = 0.008 |
| CD4/CD8 T Cells | P = 0.040 |
| | CD4+ T versus CD4/CD8 T Cells |
| TT | P = 0.005 |
| MBP | P = 0.043 |

TTEST 3

| Patients versus Controls | CD4+ T versus CD4+ T |
|---|---|
| TT | P = 0.19 |
| MBP | P = 0.10 |
| | CD4/CD8 T Cells versus |
| | CD4/CD8 T Cells |
| TT | P = 0.003 |
| MBP | P = 0.028 |

*P is significant in a reversed direction, e.g., SND Indexes are higher for foreign antigen TT than self-antigen MBP.

b). CD8+ T Cells Freshly Isolated From Most of the T1D Patients Tested Fail to Recognize Specific Target Structure HLA-E/Hsp60sp Expressed on Target Cells Compared with Normal Controls

Purified CD8+ T cells from T1D patients were tested on HLA-E transfectants B721/E loaded with Hsp60sp, B721/E loaded with B7sp or non-HLA-E binding peptide served as controls. As shown in FIG. 2B, while CD8+ T cells from normal controls specifically inhibited the B721/E loaded with Hsp60sp, but not control peptides (C), CD8+ T cells from a T1D patient (P) failed to inhibit B721/E loaded with Hsp60sp. In this regard, in 9 out of 10 T1D patients tested, CD8+ T cells from freshly isolated PBMC failed to specifically inhibit B721/E loaded with Hsp60sp, compared with normal controls (P<0.001), precisely correlating with the failure of self/nonself discrimination in each patient as shown by FIG. 3C and the results are summarized in Table 4.

TABLE 4
CD8+ T Cells In Freshly Isolated PBMC From
Majority Of The T1D Patients Tested Fail To Recognize
Specific Target Structure HLA-E/Hsp60sp Expressed
On Target Cells Compared With Normal Controls.

| | |
|---|---|
| Control | Patient |

| | Max | Max | Max | Max |
|---|---|---|---|---|
| Exper- | Inhibition | Inhibition | Inhibition | Inhibition |
| iment# | (%) | (E/T ratio) | (%) | (E/T ratio) |
| 1 | 17.2 | 0.12:1 | 0 | 3.0:1 |
| 2 | 18.0 | 0.12:1 | −5.6 | 3.0:1 |
| 3 | 14.7 | 0.12:1 | −2.8 | 3.0:1 |
| 4 | 17.1 | 0.12:1 | 18.2 | 0.12:1 |
| 5 | 22.3 | 0.24:1 | 2.0 | 3.0:1 |
| 6 | 16.8 | 0.12:1 | −1.7 | 3.0:1 |
| 7 | 22.0 | 0.12:1 | 1.0 | 3.0 1 |
| 8 | 22.2 | 0.12:1 | 2.2 | 3.0 1 |
| 9 | 18.3 | 0.12:1 | 3.6 | 3.0:1 |
| 10 | 19.0 | 0.12:1 | 2.8 | 3.0:1 |

P = 0.0000004 (4.1E−07)
P = 0.0000000287 (2.87E−10) (w/o Patient #4)

Purified CD8+ T cells freshly isolated from PBMC of the T1D patients were tested for the specificity to recognize HLA-E/Hsp60sp presented by HLA-E expression cell line B721/E in a “CD8+ T cell inhibition assay” as described. This table summarizes data from 10 T1D patients with normal controls, representative of two tests for each patient.

Taken together, a defect in the HLA-E restricted CD8+ T cell mediated regulatory pathway in majority of the T1D patients tested was detected and confirmed by two functional assays above. These results strongly suggest that HLA-E restricted CD8+ T cells participate in the immunopathogenesis of human T1D.

### The CD8+ T Cells From Most T1D Patients Tested Could be Boosted In Vitro to Restore Their Function

It was confirmed if the CD8+ T cells from T1D patients could be in vitro boosted to restore their function. Briefly, the CD8+ T cells from the T1D patients were in vitro primed with DCs loaded with either Hsp60sp [CD8(H)], B7sp [CD8(B)] or no peptide loaded [CD8(N)] as described above. The function of the boosted in vitro CD8+ T lines were tested for the specificity and the function of self/nonself discrimination using the two assays described above.

Representatively shown in FIG. 3Ac and 3Ad, CD8+ T cell lines generated from 8 out of 9 T1D patients originally tested with the defect in their CD8+ T cells restored the function of self/nonself discrimination after in vitro specifically boosted by the DCs loaded with Hsp60sp, compared with the CD8+ T cell lines generated from normal controls (FIG. 3Aa and 3Ab).

To verify the restored function of the CD8+ T cells, these CD8+ T cell lines were also tested in a CD8+ T cell inhibition assay on HLA-E transfectants B721/E loaded with Hsp60sp for their specificity. CD8(B) line from each patient served as control. Representatively shown in FIG. 3B, CD8(H) from a T1D patient specifically inhibited B721/E loaded with Hsp60sp, as effective as the normal healthy individual, but not B7sp or control peptide. CD8(B) from the same patient did not inhibit at all. This phenomenon has also been observed in above 8 out of 9 T1D patients tested who had defect on their CD8+ T cells. However, the function of the CD8+ T cells in 1 out of 9 T1D patients (#7) cannot be restored by just a simple “boost” in vitro. The restoration of the function of HLA-E restricted CD8+ T cells in most of the T1D patients, detected by CD8+ T cell inhibition assay, was confirmed by the statistical analysis that there is no differences between T1D group versus normal control group [P=0.98 (w/o patient #7)].

The function of the HLA-E restricted Hsp60sp specific CD8+ T cell lines generated from the T1D patients has also evaluated using SND Index as a parameter. As summarized in FIG. 3D and table 5, in the absence of the HLA-E restricted regulatory CD8+ T cells, in the two control groups, CD8(N) and CD8(B), in both T1D patients and normal controls, the SND Indexes are significantly lower in GAD responses than in TT responses (P<0.05).

TABLE 5
TTEST of responses to TT Versus To GAD in the presence
and absence of in vitro boosted HLA-E restricted CD8+
T cells between 10 T1D patients and 10 normal controls
TTEST 1

| Within T1D Patients | TT versus GAD |
|---|---|
| CD8(N) | *P = 0.014 |
| CD8(B) | *P = 0.025 |
| CD8(H) | P = 0.041 |
| | CD8(N) versus CD8(H) |
| TT | P = 0.002 |
| GAD | P = 0.039 |
| | CD8(B) versus CD8(H) |
| TT | P = 0.018 |
| GAD | P = 0.043 |

TTEST 2

| Within Normal Controls | TT versus GAD |
|---|---|
| CD8(N) | *P = 0.045 |
| CD8(B) | *P = 0.001 |
| CD8(H) | P = 0.026 |
| | CD8(N) versus CD8(H) |
| TT | P = 0.035 |
| GAD | P = 0.031 |
| | CD8(B) versus CD8(H) |
| TT | P = 0.001 |
| GAD | P = 0.032 |

TTEST 3

| Patients versus Controls | CD8(N) versus CD8(N) |
|---|---|
| TT | P = 0.256 |
| GAD | P = 0.545 |
| | CD8(B) versus CD8(B) |
| TT | P = 0.234 |
| GAD | P = 0.474 |
| | CD8(H) versus CD8(H) |
| TT | P = 0.253 |
| GAD | P = 1.0 |

*P is significant in a reversed direction, e.g., SND Indexes are higher for foreign antigen TT than self-antigen GAD.

However, when CD8(H) cells were added into the T cell cultures, in all the normal controls and 9 out of 10 patients, except #7 patient, the SND Indexes become significantly higher in GAD responses than in TT responses (P<0.05), indicating an inhibited immune response to self-antigen associated with an increased immune response to foreign antigen under the control of the HLA-E restricted CD8+ T cells. This notion was further confirmed by the statistical analysis that there were significant differences between responses to TT and GAD in the presence of CD8(H) within both T1D (P<0.05) and control (P<0.05) groups. However, when the same values were compared between the T1D group and control group, no significant differences were seen, P=0.25(TT) and P=1.0(GAD) respectively, showing the restored function of the HLA-E restricted CD8+ T cells in the T1D group compared with normal control group (Table 6). These observations are consistent with the notion that the defect of the CD8+ T cells can be corrected via an in vitro boost with DCs loaded with Hsp60sp in most of the T1D patients tested. Furthermore, the data also show that SND Indexes can be used as a straightforward, simple and reliable diagnostic parameter to evaluate the function of the HLA-E restricted CD8+ T cells in T1D patients in clinical settings.

TABLE 6
CD8+T Cells From Most Diabetic Patients Tested
Regain The Capacity To Recognize The Common Surrogate
Target Structure After An In Vitro Boost.

| | |
|---|---|
| Control | Patient |

| | Max | Max | Max | Max |
|---|---|---|---|---|
| Exper- | Inhibition | Inhibition | Inhibition | Inhibition |
| iment# | (%) | (E/T ratio) | (%) | (E/T ratio) |
| 1 | 17.0 | 0.12:1 | 14.8 | 0.12:1 |
| 2 | 22.6 | 0.12:1 | 23.6 | 0.12:1 |
| 3 | 17.9 | 0.12:1 | 19.4 | 0.12:1 |
| 4 | 17.1 | 0.12:1 | 18.2 | 0.12:1 |
| 5 | 17.1 | 0.24:1 | 18.7 | 0.12:1 |
| 6 | 16.9 | 0.12:1 | 17.8 | 0.12:1 |
| 7 | 19.8 | 0.12:1 | 2.1 | 3.0:1 |
| 8 | 18.4 | 0.12:1 | 20.2 | 0.12:1 |
| 9 | 28.7 | 0.12:1 | 26.2 | 0.24:1 |
| 10 | 21.8 | 0.24:1 | 18.9 | 0.12:1 |

P = 0.468
P = 0.98 (w/o patient #7)

In vitro boosted CD8(H) lines from each patient were tested for the specificity to recognize HLA-E/Hsp60sp presented by HLA-E expression cell line B721/E in a “CD8+ T cell inhibition assay” as described. This table summarizes data from 10 T1D patients with normal controls, representative of two tests for each patient

### Discussion

Self/nonself discrimination and controlling the magnitude and class of immune responses are two equally important but distinct peripheral regulatory mechanisms that function in concert to ensure an optimal function of the immune system (12, 16, 17). Currently, almost all the identified peripheral regulatory mechanisms function by controlling the magnitude and class of immune responses (16, 17), which would not be a biological basis for the development of precise and safe therapeutic approaches to prevent and treat human auto immune diseases caused by failure of self/nonself discrimination (11, 12). Looking for new conceptual framework for innovative approaches to deal with immunologically relevant clinical problems becomes more and more necessary and challenging in medicine (11, 12). In this regard, Qa-1/HLA-E restricted CD8+ T cells represent the only currently identified mechanism that function to discriminate self from nonself in the periphery (10, 11, 12, 17, 22, 23), which could lead to a new direction for specific and effective approaches to treat human auto-immune diseases.

In current studies we provide experimental evidence that the human counterpart of Qa-1 restricted CD8+ T cells, i.e. the HLA-E restricted CD8+ T cells, function to discriminate self from nonself and do exist and operate in humans.

HLA-E restricted regulatory CD8+ T cell lines can be generated by priming purified CD8+ T cells from PBMC of healthy individuals with autologous dentritic cells loaded with Hsp60sp peptide in vitro. Such cells specifically suppress the HLA-E expressing cells loaded with Hsp60sp but not loaded with control peptides. They function to discriminate self from nonself by suppressing the overall immune responses to self-antigens but not to foreign antigens.

The current studies also confirm that the HLA-E restricted CD8+ T cell mediated pathway is involved in the development and control of human auto-immune disease T1D. Compared with healthy normal individuals, data from 10 T1D patients showed that in the majority of the T1D patients tested, there is a defect in HLA-E restricted CD8+ T cell mediated pathway and most of which could be corrected by an in vitro boost with autologous DCs loaded with Hsp60sp peptide.

One of the central aspects of the HLA-E restricted CD8+ T cell mediated pathway is the unique specificity of the regulation. The specificity of the regulation is not at the level of antigens that activate the target T cells but at the level of the particular common surrogate antigen structure, HLA-E/Hsp60sp, preferentially expressed on the target T cells as a function of intermediate activation, which could be initiated by any antigens. This feature of the HLA-E restricted CD8+ T cells enables the immune system to employ a unified and simple mechanism, at a biological system level, to discriminate self from nonself, in order to control any auto-immune diseases that are caused by the defect of peripheral self/nonself discrimination. People with defects of this regulatory pathway would be prone to any organ-specific autoimmune diseases. However, the individual patient is usually dominated by one particular organ specific autoimmune disease associated with elevated reactivity to many other self-antigens (see below). This is consistent with our current observations in the T1D patients that their CD8+ T cells are incapable of inhibiting the immune responses not only to the T1D-relevant self-antigen GAD, but also to a T1D-irrelevant self-antigen MBP, while these cells also fail to recognize HLA-E/Hsp60sp presented by the target cells.

Defects of self/non-self discrimination at either central or peripheral levels could cause pathogenic autoimmunity in the periphery with distinct characteristic features. In this regard, different from global and often lethal pathogenic autoimmunity caused by the defect of central thymic negative selection, the commonly seen organ specific autoimmune diseases usually occur after the thymic negative selection is completed and are caused by defects of peripheral regulation of self/nonself discrimination and triggered by the environmental insults (12). It is expected that in order for an organ-specific autoimmune disease to occur, two hits are necessary (1) dysfunction of peripheral T cell regulation and (2) a rapid, high level presentation of particular self-antigen to the intermediate avidity self-reactive T cells, which is usually a consequence of large amount of self-antigens released from damaged cells at the site of inflammation, caused by infections or injuries. Thus, the former provides a condition for the development of organ specific auto-immune diseases and the latter, which could be influenced by certain genetic makeup of each individual and particular environmental factors, such as certain geographic locations and climates, determines which organ-specific autoimmune disease would likely to occur. Because infections or injuries are usually confined within the organ affected, an active auto-immunity evoked by such insults, from a biologically normal peripheral T cell repertoire, is likely organ-specific, which differs from the more clustered and lethal pathogenic autoimmunity caused by the defect of thymic negative selection. However, in certain cases, if the infection or injury occurs in multiple organs, auto-immunity in more than one organ could be observed in such patients. This is consistent with the observation that other autoimmune diseases were diagnosed in some T1D patients, e.g. autoimmune thyroid disease, celiac disease, and Addison's disease.

Because lack of the regulation only provides a condition for the development of auto-immune diseases, the “Avidity Model” answers a basic question that has been, in the field of immune-regulation, confusing the understanding of peripheral regulation of self/nonself discrimination, i.e. why complete knockout of the regulatory pathway does not render spontaneous development of organ specific autoimmune diseases in animals. It explains why Qa-1 KO mice do not spontaneously develop unprovoked autoimmune disease in the first instance (24). Thus, in comparison with WT mice, although Qa-1 KO mice do require an experimental induction, like in the WT mice, to develop the first episode of EAE, they lost the capacity to resist the re-induction of EAE due to the defect of Qa-1 restricted CD8+ T cells (24). The phenotype of the Qa-1 KO mice precisely reveals how peripheral self-tolerance is maintained in a biological context of the most commonly seen cases in real life, compared with the control of rarely seen, drastic and lethal global pathogenic auto-immunity that is primarily caused by genetic defect of thymic negative selection (11, 12). The biological significance of HLA-E restricted CD8+ T cells in maintaining peripheral self-tolerance has also been demonstrated in our current studies. In all the tests performed with different settings in normal controls, the immune responses of CD4+ T cells to self-antigens are always significantly higher (P<0.05) or the same compared with immune responses to foreign antigens in the absence of the HLA-E restricted CD8+ T cells. In contrast, in all the tests containing the HLA-E restricted CD8+ T cells, the immune responses of CD4+ T cells to self-antigens are always significantly lower (P<0.05) compared with immune responses to foreign antigens without any exceptions. This notion is also supported by the fact that majority of the T1D patients tested have lost the function of their HLA-E restricted CD8+ T cells.

In the current studies, among the 10 T1D patients tested, 9 patients showed the defect of HLA-E restricted CD8+ T cells with one exception of patient #4. This unexpected but consistent observation of high incidence of the defect of the HLA-E restricted CD8+ T cells in T1D patients tested makes the postulation that HLA-E restricted CD8+ T cell mediated pathway plays a major role in the development and control of human T1D. However, T1D in a much smaller portion of patients could be caused by defect of different regulatory mechanisms other than the HLA-E restricted CD8+ T cells. Thus, there might be two major categories of T1D in humans, dependent or independent of the defect of HLA-E restricted CD8+ T cells, which require distinct approaches for the treatment. Furthermore, among the 9 T1D patients with defect of HLA-E restricted CD8+ T cells, CD8+ T cells from 8 patients could regain the function after an in vitro boost with autologous DCs loaded with Hsp60sp. However, the function of CD8+ T cells from the patient could not be corrected by such an in vitro boost. This observation indicates that the defect of the CD8+ T cell mediated pathway in this patient may not at the level of the CD8+ T cell itself, but at the level of either dendritic cell which may not be capable of inducing the CD8+ T cells, or CD4+ T cell which may not be susceptible to the CD8+ T cell regulation. Further systematical studies should be performed to investigate these possibilities.

The two major assays established and used in current studies are particularly designed to detect the HLA-E restricted CD8+ T cells. The first “CD8+ T cell inhibition assay” is to detect the specificity of the HLA-E restricted CD8+ T cells for targeting intermediate but not high avidity T cells based on the fact that the preferential expression of HLA-E/Hsp60sp complex only occurs in intermediate avidity T cells (14). The second is a T cell proliferation assay to systematically test self/nonself discrimination of the human CD8+ T cells by measuring and comparing the overall avidity of T cell immune responses to self versus to foreign antigens, in the presence and absence of HLA-E restricted CD8+ T cells (15).

Combination of these two assays provides a unique and reliable assay system that enables the identification of the specificity and function of human HLA-E restricted CD8+ T cells in both basic and clinical studies. Compared with currently popular assays used to detect Foxp3+ T regs, this assay system has major advantages. For example, in the assays used to detect the function of Foxp3+ Tregs, target T cells are usually activated nonspecifically by agents like the anti-CD3 mAb or mitogens. This is because the specific target structure and the particular T cell target population that are recognized by the Foxp3+ T reg have never been precisely identified, representing a major unsolved issue in the biology of Foxp3+ Tregs (11, 12, 17). In contrast, the CD8+ T cell inhibition assay can unequivocally determine if the CD8+ T cells are HLA-E restricted and if these cells specifically recognize the particular target structure HLA-E/Hsp60sp expressed on the target cells while self/nonself discrimination assay can precisely test the biological function of these cells. As importantly, the biological effectiveness of the degree and magnitude of the regulation, detected by these two assays, has not only been confirmed by the statistical analysis (Table 3), but also supported by the observations that such degree and magnitude of the regulation is sufficient to effectively protect animals from T1D and EAE (15).

Taken together, our current findings not only could open up a new theme to understand the biological basis of peripheral self-tolerance and control of human auto-immune diseases based on the conceptual framework of the “Avidity Model”, but also provide a potentially clinical approach to specifically and effectively prevent and treat human T1D at both diagnostic and therapeutic levels. Identification of the peripheral regulatory mechanisms, that function to discriminate self from non-self, opens up new possibilities of novel clinical interventions to prevent and treat autoimmune diseases without damaging the anti-infection and anti-tumor immunity, which is the major side-effect of the currently used immuno-therapeutic drugs. Based on our current findings, the potential treatments could be either vaccinating the patients with autologous DCs loaded with Hsp60sp or transfer the in vitro boosted CD8+ T cells back to the patients. However, at current stage, we do not know if the effect of boosting the HLA-E restricted CD8+ T cells in vitro could be directly translated into an in vivo therapy to treat human T1D. In this regard, the potential use as a therapeutic approach could be immediately explored further in vivo using murine T1D model in NOD mice, although murine T1D studies in NOD mice sometimes could not provide direct correlation with human T1D. Furthermore, our assay system allows not only evaluation of the functional status of the HLA-E restricted CD8+ T cells in established T1D patients but also early diagnosis for symptom free “pre-T1D patients”, siblings of T1D patients who have the potential to develop full-blown T1D later.

### Multiple Sclerosis

The specificity assay and self/non-self discrimination assay was performed on samples from three multiple sclerosis (MS) patients, with two normal subjects as controls. All three MS patients showed the defect in their HLA-E restricted CD8+ T cells of being unable to discriminate self from non-self.

### Blind Study

Of six subjects blind tested, five were identified to have a self/non-self recognition defect in the HLA-E restricted CD8+ T cells and were confirmed as having autoimmune disease symptoms. One subject's sample showed the defect in the HLA-E restricted CD8+ T cells even though the subject has no clinical symptoms. The subject will be followed to determine if an automimmune disease develops.

In the blind sample a defect of the HLA-E restricted CD8+ T cells from a type 1 diabetes (T1D) patient whose blood tests were negative for all autoantibodies tested, the specificity assay and self-nonself discrimination assay could definitively diagnose an auto-immune disease where no other assay was available. This can serve as early diagnosis to identify high-risk populations who can be treated for correction of the regulatory pathway defect to prevent the subsequent development of autoimmune diseases.

### Material and Methods

### Reagents

Anti-HLA-E mAb 3D-12 and Control mAb 4D-12 are a kind gift from Dr. Daniel Geraghty (Fred Hutchinson Cancer Research Center, Seattle, USA). The staining reagents, Fluorescein (Fl)-anti human CD8), Phycoerythorine (PE)-anti human CD4) and Fluorescein (FI)-goat anti-mouse were purchased from B D Pharmingen, N.J., USA. Peptides: human HSP60sp (QMRPVSRVL) (SEQ ID NO:1); hB7sp (VMAPRTVLL) (SEQ ID NO:3); TT-830(QYIKANSKFIGITE) (SEQ ID NO:4); GAD555-567(NFFRMVISNPAAT) (SEQ ID NO:5); MBP84-102 (NPVVHFFKNIVTPRTPPP) (SEQ ID NO:6) are synthesized by GeneScript Corporation, N.J., USA. PPD was purchased from Sanofi Pasteur Limited, Toronto, Canada.

### Preparation of Human PBMC and Purified CD8+ and CD4+ T Cells

PBMC were prepared from heparinized blood, which was diluted 1:1 with Hank's balanced salt solution and layered over Ficoll-Hypaque, and centrifuged at 1,000 g for 30 min in the Sorvall RC-3 (Ivan Sorvall, Inc., N.Y., USA). The lymphocytes at the serum Ficoll interface were removed and washed three times and ready for further purification of subsets. Both CD8+ and CD4+ T cells in all experiments presented in this study were positively selected by MACS magnetic beads (Miltenyi Biotec, Inc. Auburn Calif., USA) as described (13). Briefly, the PBMC were incubated with anti Human CD4 or CD8 conjugated magnetic beads at 10×10⁶ cells /10 ul of beads and the CD+ and CD− population were isolated using a separation column exposed to a magnetic field according to the manufacturer's protocol. The purity of the CD4+ or CD8+ T cells was >95%.

### Generation of HLA-E Expression Transfectants

To derive a DSRed-HLA-E Fusion Construct, the cDNA encoding human HLA-E was isolated by reverse transcription PCR using total RNA derived from the human B cell line, B721. The following primer pair was used for amplification: forward—GGGGATCCAACAAGCTGTGAGACTCAGACCC (SEQ ID NO:7). The resulting PCR product was subcloned into the vector pCR2.1 (Invitrogen Corp., now Life Technologies, Calif., USA) and amplified clones were fully sequenced on both strands to verify identity. Six independent full-length clones yielded sequence identical to that published for the HLA-E 101 haplotype, except that all clones lacked the 3′ termination codon by design. Sequence-confirmed clones were excised from pCR2.1 using restriction sites designed into the primers (XhoI, 5′ end; BamHI, 3′ end) and subcloned into the mammalian expression vector pDSRed-Express-N1 (Clonetech, Inc., Calif., USA) using the same restriction sites. The insertion of the HLA-E cDNA into the pDSRed vector yielded a single open reading frame encoding a fusion protein consisting of HLA-E joined to a variant of the discosoma species red fluorescent protein (Clonetech, Inc., Calif., USA). Expression of fluorescent HLA-E was confirmed by a transient expression assay in the 293T embryonic kidney fibroblast cell line. The pDS-Red-HLA-E construct was subsequently introduced into a human HLA-A, B, C deficient B cell line B721 (18), by electroporation, and stable expression was obtained following 3 weeks of selection for vector-encoded neomycin resistance using Geneticin® (G418) (Gibco BRL Life Technologies, Inc,. Calif., USA) and the stable transfectants were obtained by further subcloning procedure.

### Testing HLA-E Surface Expression of HLA-E Transfectants

The surface expression of HLA-E on B721 transfected with HLA-E was assessed by exogenously loading the cells with hHsp60sp and B7sp or control non-HLA-E binding peptides at 26° C. for 18 hours. Cells were then washed, stained with anti-HLA-E mAb 3D-12 (21) followed by F-goat anti-mouse Ig, and analyzed on a FACScan flow cytometer and CellQuest™ software (Becton Dickinson, Mountain View, Calif., USA) as previously described (13). Mab 4D-12 served as control (21).

### Generation of HLA-E Restricted Hsp60sp Specific CD8+ T Cell Lines

Dendritic cells were derived from PBMC depleted of CD4+ and CD8+ T cells and were cultured in 6 well plates in serum free click's medium, at 37° C., 5% CO2 for 1-1.5 hours. The cells were then washed and cultured for 6 days in media containing GM-CSF and IL-4 at final concentrations of 80 ng/ml and 20 ng/ml respectively. DCs are harvested on D6 and loaded with either Hsp60sp or B7sp, at 50 uM, 37° C., for 2 hours. The 1.5-2×10⁶ of purified CD8+ T cells were then co-cultured with 0.5×10⁶ peptide loaded DCs in 1 ml in 48 well plate to set up the lines of CD8(H), CD8(B) and CD8(N) (primed by DC without loading with peptide). IL-2 is added on the second day. These three types of lines have been established for each T1D patient and control throughout this study.

### CD8+ T Cell Inhibition Assay

This type of assay is to detect the specificity of the Qa-1 or HLA-E restricted CD8+ T cells for targeting intermediate but not high avidity T cells based on the fact that the preferential expression of Qa-1 or HLA-E/Hsp60sp complex only occurs in intermediate avidity T cells (14, 15). Briefly, CD8(H) and CD8(B) lines were generated as described. HLA-E transfected cells (B721/E) established were passively loaded with testing peptides overnight at 26° C. Equal number of unlabeled B721/E cells loaded with peptides and CFSE labeled B721 cells that are not loaded with peptide were mixed and a graded number of CD8(H) cells were added to the targets from E/T ratio 3:1 to 0.01:1. CD8[B] cells loaded with different peptides serve as additional controls. In this regard, we have established that CD8(H) cells tested have no effect on B721 cells alone or B721 cells pulsed with hsp60sp or B7sp. 5-6 days later, the cell mixtures were assessed by FACS analysis in which the CD8+ T cells were gated out during the analysis. The ratio between two types of targets was calculated to evaluate the effect of testing CD8+ T cells on the targets. The ratio between peptide-loaded (non-CFSE-labeled) B721/E cells and non-loaded (CFSE labeled) B721 cells in the presence of CD8+ T cells is determined as % of specific inhibition: {[the ratio of loaded B721/E versus unloaded B721 cells in control cultures (without CD8+ T cells)—the ratio in experimental cultures with CD8+ T cells]/the ratio in control cultures}×100% (14, 15)

### Detection of Intracellular Cytolytic Enzymes (CE) Secreted by the CD8+ T Cells

CD8(H) and CD8(B) lines were generated from healthy individuals as described. The established HLA-E transfected cells (B721/E) served as targets to trigger the CD8+ T cells, and were passively loaded with Hsp60sp peptide overnight at 26° C. and the B7sp peptide served as control. A graded number of testing CD8(H) and CD8(B) lines were added to the target B721/E cells loaded with different peptides from E/T ratio 3:1 to 0.005:1. At different time points, three color intracellular staining was performed on the cell mixture with anti-Perforin-PE, anti-Granzyme A-FITC and anti-Granzyme B-Bio/Cy following manufacture instructions (B D Pharmingen, San Diego, Calif., USA). The cells were assessed by FACS analysis in which the CD8+ T cells were gated in during the analysis. The CE Expression Index was calculated as a function of different E/T ratio: [(% of double positive CE stained CD8+ T cells from different E/T ratio cultures)—(% of double positive CE stained CD8+ T cells from the CD8+ T cells that were not be triggered by the target cells)]/% of double positive CE stained CD8+ T cells from the CD8+ T cells that were not be triggered by the target cells.

### Self/Nonself Discrimination Assay

A functional assay to systematically test the effect of the human CD8+ T cell lines on the overall immune responses of CD4+ T cells to self-antigens versus to foreign antigens has been established by measuring the overall avidity of T cell immune response to different antigens in a standard T cell proliferation assay. In this regard, we have chosen TT and PPD as foreign antigens and GAD and MBP as self-antigens, respectively, in our standard protocols. Briefly, purified CD4+ T cells were activated, in the presence of irradiated splenic cells as APCs by a series dose, ranging from 0.08 to 50 uM, (PPD was used ranging from 1:12,500 to 1:20) of either GAD (or MBP) or TT (or PPD) for 24 hours. Antigens were washed away and the mixture of 0.1×10⁵ purified CD4+ T cells plus 1×10⁵ irradiated splenic cells were plated into round bottom 96 well plates in AIM-V serum free lymphocyte medium (GIBCO) supplemented with L-glutamine at 1 mM. CD8(H) were also added to the CD4+ T cells in a E/T ratio of 0.1-0.2:1 and further cultured for additional 5-6 days. In this regard, we had titrated the amount of the CD8(H) cells added in to the CD4+ T cell culture activated by different antigen doses and 10-20% of the CD8+ T cells adding into CD4+ T cell culture was chosen to reveal optimal inhibition effect of the CD8(H) cells. CD8(B) and CD8(N) served as controls. During the last 18 hours of 5-6 day culture, 3H thymidine was added (1 uCi/well) and incorporation of labeling was measured by liquid scintillation counting. Cell proliferation, as counts per minute, has been plotted against antigen concentration, and ED₅₀value was derived by calculating the intercept of antigen concentration leading to half maximum proliferation (32, 33). In this regard, it is well accepted that “ED₅₀” is currently the most reliable approach to assess the avidity of antigen specific T cell clones, which reflects an integrated function of direct ligation of the TCR with MHC/peptide complexes as well as signaling via co-stimulatory molecules. We thus choose this approach to measure the overall avidity of a T cell response, which has been successfully applied in our previous studies (13-15). We also designed a new parameter to evaluate the function of self/nonself discrimination of the T cells, in addition to “ED₅₀”, the self/nonself discrimination Index: antigen doses that elicit the highest T cell proliferation between immune response to self-antigens versus to foreign antigens in each individual, as described in details in the text.

### REFERENCES

1. Kappler, J. W., N. Roehm, and P. Marrack. 1987. T cell tolerance by clonal elimination in the thymus. Cell 49:273-280.
2. Hengartner, H., B. Odermatt, R. Schneider, M. Schreyer, G. Walle, H. R. MacDonald, and R. M. Zinkernagel. 1988. Deletion of self-reactive T cells before entry into the thymus medulla. Nature 336:388-390.
3. Pircher, H., U. H. Rohrer, D. Moskophidis, R. M. Zinkernagel, and H. Hengartner. 1991. Lower receptor avidity required for thymic clonal deletion than for effector T-cell function. Nature 351:482-485.
4. Bouneaud, C., P. Kourilsky, and P. Bousso. 2000. Impact of negative selection on the T cell repertoire reactive to a self-peptide: a large fraction of T cell clones escapes clonal deletion. Immunity 13:829-840.
5. Sandberg, J. K., L. Franksson, J. Sundback, J. Michaelsson, M. Petersson, A. Achour, R. P. Wallin, N. E. Sherman, T. Bergman, H. Jornvall, D. F. Hunt, R. Kiessling, and K. Karre. 2000. T cell tolerance based on avidity thresholds rather than complete deletion allows maintenance of maximal repertoire diversity. J Immunol 165:25-33.
6. Jiang, H., S. Curran, E. Ruiz-Vazquez, B. Liang, R. Winchester, and L. Chess. 2003. Regulatory CD8+ T cells fine-tune the myelin basic protein-reactive T cell receptor V beta repertoire during experimental autoimmune encephalomyelitis. Proc Natl Acad Sci USA 100:8378-8383. Epub 2003 Jun 8324.
7. Anderton, S. M., C. G. Radu, P. A. Lowrey, E. S. Ward, and D. C. Wraith. 2001. Negative selection during the peripheral immune response to antigen. J Exp Med 193:1-11.
8. Han, B., P. Serra, J. Yamanouchi, A. Amrani, J. F. Elliott, P. Dickie, T. P. Dilorenzo, and P. Santamaria. 2005. Developmental control of CD8 T cell-avidity maturation in autoimmune diabetes. J Clin Invest 115:1879-1887.
9. Zehn, D., and M. J. Bevan. 2006. T cells with low avidity for a tissue-restricted antigen routinely evade central and peripheral tolerance and cause autoimmunity. Immunity 25:261-270.
10. Jiang, H., and L. Chess. 2000. The Specific Regulation of Immune Responses by CD8+ T Cells Restricted by the MHC Class IB Molecule, QA-1. Annu. Rev. Immunol. 18:185-216.
11. Jiang, H., and L. Chess. 2008. Qa-1/HLA-E-restricted regulatory CD8+ T cells and self-nonself discrimination: An essay on peripheral T-cell regulation. Hum Immunol 69:721-727.
12. Jiang, H., and L. Chess. 2009. How the Immune System Achieves Self Non-self Discrimination During Adaptive Immunity. Advances in Immunology 102:94-133.
13. Jiang, H., Y. Wu, B. Liang, Z. Zheng, G. Tang, J. Kanellopoulos, M. Soloski, R. Winchester, I. Goldstein, and L. Chess. 2005. An affinity/avidity model of peripheral T cell regulation. J. Clin. Invest. 115:302-312.
14. Chen, W., L. Zhang, B. Liang, Y. Saenger, J. Li, L. Chess, and H. Jiang. 2007. Perceiving the avidity of T cell activation can be translated into peripheral T cell regulation. Proc Natl Acad Sci USA 104:20472-20477.
15. Wu, Y., Z. Zheng, Y. Jiang, L. Chess, and H. Jiang. 2009. The Specificity of T Cell Regulation That Enables Self Non-Self Discrimination In The Periphery. Proc Natl Acad Sci USA 106:534-539.
16. Cohn, M. 2004. Whither T-suppressors: if they didn't exist would we have to invent them? Cell Immunol 227:81-92.
17. Jiang, H., and L. Chess. 2006. Regulation of immune responses by T cells. N Engl J Med 354:1166-1176.
18. Lee, N., D. R. Goodlett, A. Ishitani, H. Marquardt, and D. E. Geraghty. 1998. HLA-E surface expression depends on binding of TAP-dependent peptides derived from certain HLA class I signal sequences. J Immunol 160:4951-4960.
19. Lee, N., M. Llano, M. Carretero, A. Ishitani, F. Navarro, M. Lopez-Botet, and D. E. Geraghty. 1998. HLA-E is a major ligand for the natural killer inhibitory receptor CD94/NKG2A [see comments]. Proc Natl Acad Sci USA 95:5199-5204.
20. Michaelsson, J., C. Teixeira de Matos, A. Achour, L. L. Lanier, K. Karre, and K. Soderstrom. 2002. A signal peptide derived from hsp60 binds HLA-E and interferes with CD94/NKG2A recognition. J Exp Med 196:1403-1414.
21. Ishitani, A., N. Sageshima, N. Lee, N. Dorofeeva, K. Hatake, H. Marquardt, and D. E. Geraghty. 2003. Protein expression and peptide binding suggest unique and interacting functional roles for HLA-E, F, and G in maternal-placental immune recognition. J Immunol 171:1376-1384.
22. Jiang, H., and L. Chess. 2004. An integrated model of immunoregulation mediated by regulatory T cell subsets. Adv Immunol 83:253-288.
23. Jiang, H. 2005. The Qa-1 dependent CD8+ T cell mediated regulatory pathway. Cell Mol Immunol 2:161-167.
24. Hu, D., K. Ikizawa, L. Lu, M. E. Sanchirico, M. L. Shinohara, and H. Cantor. 2004. Analysis of regulatory CD8 T cells in mice deficient in the Qa-1 class Ib molecule, H2-T23. Nat. Immunol. 5:516-523.
25. Chaput, N., N. E. Schartz, F. Andre, J. Taieb, S. Novault, P. Bonnaventure, N. Aubert, J. Bernard, F. Lemonnier, M. Merad, G. Adema, M. Adams, M. Ferrantini, A. F. Carpentier, B. Escudier, T. Tursz, E. Angevin, and L. Zitvogel. 2004. Exosomes as potent cell-free peptide-based vaccine. II. Exosomes in CpG adjuvants efficiently prime naive Tc1 lymphocytes leading to tumor rejection. J Immunol 172:2137-2146.
26. Dal Porto, J., T. E. Johansen, B. Catipovic, D. J. Parfiit, D. Tuveson, U. Gether, S. Kozlowski, D. T. Fearon, and J. P. Schneck. 1993. A soluble divalent class I major histocompatibility complex molecule inhibits alloreactive T cells at nanomolar concentrations. Proc Natl Acad Sci USA 90:6671-6675.
27. Oelke, M., and J. P. Schneck. 2004. HLA-Ig-based artificial antigen-presenting cells: setting the terms of engagement. Clin Immunol 110:243-251.
28. Casares, S., C. A. Bona, and T. D. Brumeanu. 1997. Engineering and characterization of a murine MHC class II-immunoglobulin chimera expressing an immunodominant CD4 T viral epitope. Protein Eng 10:1295-1301.
29. Malherbe, L., C. Filippi, V. Julia, G. Foucras, M. Moro, H. Appel, K. Wucherpfennig, J. C. Guery, and N. Glaichenhaus. 2000. Selective activation and expansion of high-affinity CD4+ T cells in resistant mice upon infection with Leishmania major. Immunity 13:771-782.
30. Casares, S., C. A. Bona, and T. D. Brumeanu. 2001. Enzymatically mediated engineering of multivalent MHC class II-peptide chimeras. Protein Eng 14:195-200.
31. Altman, J. D., P. A. Moss, P. J. Goulder, D. H. Barouch, M. G. McHeyzer-Williams, J. I. Bell, A. J. McMichael, and M. M. Davis. 1996. Phenotypic analysis of antigen-specific T lymphocytes. Science 274:94-96.
32. Fasso, M., N. Anandasabapathy, F. Crawford, J. Kappler, C. G. Fathman, and W. M. Ridgway. 2000. T cell receptor (TCR)-mediated repertoire selection and loss of TCR vbeta diversity during the initiation of a CD4(+) T cell response in vivo. J Exp Med 192:1719-1730.
33. Targoni, O. S., and P. V. Lehmann. 1998. Endogenous myelin basic protein inactivates the high avidity T cell repertoire. J Exp Med 187:2055-2063.

1. A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

2. A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a B7sp peptide;
d) quantifying proliferation of the HLA-E+ cell which is loaded with the B7sp peptide and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser or equal amount of proliferation quantified in step d) than quantified in step b) indicates that the subject's CD8+ T-cells are not able to functionally recognize the HLA-E/Hsp60sp target structure.

3. The method of claim 1, wherein the sample of the subject's CD8+ T-cells in step a) and/or in step c) is a peripheral blood mononucleocyte cell sample obtained from the subject.

4. The method of claim 1, wherein the subject has an autoimmune disease.

5. The method of claim 4, wherein the autoimmune disease is type 1 diabetes.

6. A method of determining if a subject not known to have an autoimmune disease is predisposed to develop the autoimmune disease comprising determining if the subject's CD8+ T-cells are able to functionally recognize an HLA-E/Hsp60sp target structure on the surface of a cell and inhibit proliferation of the cell, wherein if the subject's CD8+ T-cells are unable to functionally recognize an HLA-E/Hsp60sp target structure on the surface of the cell and inhibit proliferation of the cell then the subject is predisposed to develop the autoimmune disease.

7. The method of claim 6, wherein whether the subject's CD8+ T-cells are able to functionally recognize an HLA-E/Hsp60sp target structure on the surface of the cell and inhibit proliferation of the cell is determined by:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step c); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser amount of proliferation quantified in step d) than quantified in step b) indicates that the subject's CD8+ T-cells are not able to functionally recognize the HLA-E/Hsp60sp target structure.

8. A method of determining if a subject not known to have an autoimmune disease is predisposed to develop the autoimmune disease comprising determining if the subject's HLA-E restricted CD8+ T-cells are able to discriminate self from non-self, wherein if the subject's HLA-E restricted CD8+ T-cells are unable to discriminate self from non-self then the subject is predisposed to develop the autoimmune disease.

9. The method of claim 8, wherein determining if a subject's CD8+ T-cells are able to discriminate self from non-self comprises:
A)
i) contacting a population of the subject's CD4+ cells with (a) an amount of a self-antigen and (b) an amount of antigen-presenting cells so as to thereby activate CD4+ cells of the population;
ii) washing the population of activated CD4+ cells so as to remove the self-antigen;
iii) culturing a portion of the population of activated CD4+ cells and amount of the population of antigen-presenting cells together in the presence of a population of the subject's CD8+ T-cells;
iv) quantifying proliferation of the activated CD4+ cells; and
v) repeating steps A)i) through A)iv) with different amounts of self-antigen so as to determine the amount of self-antigen required to elicit maximum proliferation of the activated CD4+ cells and thereby determine a self-antigen ED₅₀;
B)
i) contacting a population of the subject's CD4+ cells with (a) a foreign antigen and (b) an amount of antigen-presenting cells so as to thereby activate CD4+ cells of the population;
ii) washing the population of activated CD4+ cells so as to remove the foreign antigen;
iii) culturing a portion of the population of activated CD4+ cells from step B)ii) and amount of the population of antigen-presenting cells together in the presence of a population of the subject's CD8+ T-cells;
iv) quantifying proliferation of the activated CD4+ cells from step B)iii); and
v) repeating steps B)i) through B)iv) with different amounts of foreign antigen so as to determine the amount of foreign antigen required to elicit maximum proliferation of the activated CD4+ cells and thereby determine a foreign antigen ED₅₀;
wherein steps A) and B) can be performed in any order; and
C) comparing the self-antigen ED, and the foreign antigen ED₅₀, wherein a foreign antigen ED₅₀ greater than the self-antigen ED₅₀ indicates that the subject's CD8+ T-cells are unable to discriminate self from non-self and wherein a foreign antigen ED₅₀ equal to or lesser than the self-antigen ED₅₀ indicates that the subject's CD8+ T-cells are able to discriminate self from non-self.

10-11. (canceled)

12. A method of determining if a subject's CD8+ T-cells are able to discriminate self from non-self comprising:
A)
i) contacting a population of purified CD4+ cells with (a) an amount of a self-antigen and (b) and an amount of antigen-presenting cells so as to thereby activate CD4+ cells of the population;
ii) washing the population of activated CD4+ cells so as to remove the self-antigen;
iii) culturing a portion of the population of activated CD4+ cells and amount of the antigen-presenting cells together in the presence of a population of the subject's CD8+ T-cells;
iv) quantifying proliferation of the activated CD4+ cells; and
v) repeating steps A)i) through A)iv) with different amounts of self-antigen so as to determine the amount of self-antigen required to elicit maximum proliferation of the activated CD4+ cells and thereby determine a self-antigen ED₅₀ for the population of purified CD4+ cells;
B)
i) contacting a sample of peripheral blood mononucleocyte cells obtained from the subject with (a) the self-antigen and (b) and a population of antigen-presenting cells so as to thereby activate CD4+ cells of the population;
ii) washing the population of activated CD4+ cells so as to remove the self -antigen;
iii) culturing a portion of the population of activated CD4+ cells of step B)ii) and amount of antigen-presenting cells together in the presence of a population of the subject's CD8+ T-cells;
iv) quantifying proliferation of the activated CD4+ cells from step B)iii); and
v) repeating steps B)i) through B)iv) with different amounts of self-antigen so as to determine the amount of self-antigen required to elicit maximum proliferation of the activated CD4+ cells and thereby determine the self-antigen ED₅₀ for the sample of peripheral blood mononucleocyte cells;
wherein steps A) and B) can be performed in any order; and
C) comparing the self-antigen ED₅₀ and the foreign-antigen ED₅₀, wherein a foreign-antigen ED₅₀ greater than the self-antigen ED₅₀ indicates that the subject's CD8+ T-cells are unable to discriminate self from non-self and wherein a foreign-antigen ED₅₀ equal to or lesser than the self-antigen ED₅₀ indicates that the subject's CD8+ T-cells are able to discriminate self from non-self.

13. A method of determining if a subject's CD8+ T-cells are able to functionally recognize an HLA-E/Hsp60sp target structure comprising:
a) contacting a sample of the subject's CD8+ T-cells with a HLA-E+ cell which is loaded with Hsp60sp;
b) quantifying proliferation of the HLA-E+ cell which is loaded with Hsp60sp and contacted with the subject's CD8+ T-cells in step a);
c) contacting a sample of the subject's CD8+ T-cells with a HLA-E+ cell which is loaded with a peptide which does not bind to HLA-E;
d) quantifying proliferation of the HLA-E+ cell which is loaded with the peptide which does not bind to HLA-E and contacted with the subject's CD8+ T-cells in step a); and
e) comparing the proliferation quantified in step d) with the proliferation quantified in step b),
wherein a greater amount of proliferation quantified in step d) than quantified in step b) indicates that the subject's CD8+ T-cells are able to functionally recognize the HLA-E/Hsp60sp target structure and wherein a lesser amount of proliferation quantified in step d) than quantified in step b) indicates that the subject's CD8+ T-cells are not able to functionally recognize the HLA-E/Hsp60sp target structure.

14. The method of claim 13, wherein the sample of the subject's CD8+ T-cells in step a) and/or in step c) is a peripheral blood mononucleocyte cell sample obtained from the subject.

15. The method of claim 13, wherein the subject has an autoimmune disease.

16. The method of claim 15, wherein the autoimmune disease is type 1 diabetes.

17. The method of claim 1, wherein the subject is a human.

18. A method of determining whether a subject is likely to develop an autoimmune disorder comprising determining if a subject's CD8+ T cells are not able to discriminate self from the non-self, wherein a subject whose CD8+ T cells are not able to discriminate self from non-self is determined as likely to develop an autoimmune disease.

19. (canceled)

20. A method of determining if a subject suffering from an autoimmune disease can be treated for the autoimmune disease by administration of HSP60sp-loaded cells comprising determining if the subject's CD8+ T cells functionally recognize an HLA-E/Hsp60sp target structure, wherein a subject suffering from the autoimmune disease whose CD8+ T cells are not able to functionally recognize the HLA-E/Hsp60sp target structure is determined as treatable by administration of Hsp60sp-loaded cells.

21. (canceled)

22. A method of determining if a subject's HLA-E restricted CD8+ T cells are activated by HLA-E/Hsp60sp comprising:
a) contacting a sample comprising HLA-E restricted CD8+ T cells obtained from the subject with a composition comprising HLA-E/Hsp60sp; and
b) detecting if step (a) results in secretion of an intracellular cytolytic enzyme by an HLA-E restricted CD8+ T cell of the sample,
wherein secretion of an intracellular cytolytic enzyme by an HLA-E restricted CD8+ T cell of the sample indicates that the subject's HLA-E restricted CD8+ T cells are activated by HLA-E/Hsp60sp, and wherein no detectable secretion of an intracellular cytolytic enzyme in step b) indicates that the subject's HLA-E restricted CD8+ T cells are not activated by HLA-E/Hsp60sp.

23-27. (canceled)

28. A method to identify a functioning HLA-E restricted CD8+ T cell in a sample comprising:
a) contacting the sample with a composition comprising HLA-E/Hsp60sp; and
b) detecting if step (a) results in secretion of an intracellular cytolytic enzyme in a cell of the sample,
wherein secretion of an intracellular cytolytic enzyme in a cell of the sample indicates that the sample comprises a functioning HLA-E restricted CD8+ T cell.

29. A method for prophylactically treating a subject against developing an autoimmune disease comprising administering to the subject dendritic cells loaded with Hsp60sp peptide so as to activate HLA-E restricted CD8+ T cells in the subject and thereby prophylactically treat the subject against developing the autoimmune disease.

30. A method for treating a subject having an autoimmune disease comprising administering to the subject dendritic cells loaded with Hsp60sp peptide so as to activate HLA-E restricted CD8+ T cells in the subject and thereby treat the autoimmune disease.

31. A method of treating a subject having an autoimmune disease comprising:
a) determining if HLA-E restricted CD8+ T cells obtained from the subject are able to discriminate self from the non-self; and
b) if the subject's HLA-E restricted CD8+ T cells are determined in step a) as not able to discriminate self from the non-self, administering to the subject an exosome or dentritic cell loaded Hsp60sp peptide so as to activate HLA-E restricted CD8+ T cells in the subject and thereby treat the autoimmune disease.

32-34. (canceled)
